SEQUENCE LISTING (44 sequences)

SEQ ID NO: 1 - DNA, 22 bp, Artificial Sequence
  Synthetic
gctcccagaaccttctctattt

SEQ ID NO: 2 - DNA, 23 bp, Artificial Sequence
  Synthetic
ccaggtaatcctgagctctaatg

SEQ ID NO: 3 - DNA, 20 bp, Artificial Sequence
  Synthetic
tgcagatgctgtgggtaaat

SEQ ID NO: 4 - DNA, 21 bp, Artificial Sequence
  Synthetic
ccataaggcttccgcaataga

SEQ ID NO: 5 - DNA, 22 bp, Artificial Sequence
  Synthetic
cctgacggagaaataaggaaga

SEQ ID NO: 6 - DNA, 22 bp, Artificial Sequence
  Synthetic
cccaggagaagacacagataaa

SEQ ID NO: 7 - DNA, 22 bp, Artificial Sequence
  Synthetic
ttgattcagagcctcccattag

SEQ ID NO: 8 - DNA, 21 bp, Artificial Sequence
  Synthetic
gcgaggagtaggcttgtattt

SEQ ID NO: 9 - DNA, 20 bp, Artificial Sequence
  Synthetic
ctcagcaatcactggcatca

SEQ ID NO: 10 - DNA, 22 bp, Artificial Sequence
  Synthetic
actgggcaactccagaaataag

SEQ ID NO: 11 - DNA, 23 bp, Artificial Sequence
  Synthetic
catgtgcattctcttccaaagtc

SEQ ID NO: 12 - DNA, 20 bp, Artificial Sequence
  Synthetic
ctccttggtgaccgcattat

SEQ ID NO: 13 - DNA, 22 bp, Artificial Sequence
  Synthetic
ctcacagtccttcgacatcttc

SEQ ID NO: 14 - DNA, 20 bp, Artificial Sequence
  Synthetic
tgttgagcctcactttctgg

SEQ ID NO: 15 - DNA, 21 bp, Artificial Sequence
  Synthetic
acctcatcctccgagacttta

SEQ ID NO: 16 - DNA, 20 bp, Artificial Sequence
  Synthetic
gcactgaaactcctggtctt

SEQ ID NO: 17 - DNA, 21 bp, Artificial Sequence
  Synthetic
gacagcctatgccaacaagta

SEQ ID NO: 18 - DNA, 21 bp, Artificial Sequence
  Synthetic
ttacaggaagggcaactcatc

SEQ ID NO: 19 - DNA, 20 bp, Artificial Sequence
  Synthetic
gctggtaaccgggattatgt

SEQ ID NO: 20 - DNA, 21 bp, Artificial Sequence
  Synthetic
gcaggaacatgaggaggtaat

SEQ ID NO: 21 - DNA, 22 bp, Artificial Sequence
  Synthetic
acagatgctgctgactgatatg

SEQ ID NO: 22 - DNA, 22 bp, Artificial Sequence
  Synthetic
aagatagtgggtggtaggagaa

SEQ ID NO: 23 - DNA, 21 bp, Artificial Sequence
  Synthetic
gcgtgggatagagttgacatt

SEQ ID NO: 24 - DNA, 21 bp, Artificial Sequence
  Synthetic
tcacaagggcatccaacatag

SEQ ID NO: 25 - DNA, 21 bp, Artificial Sequence
  Synthetic
ggtaacagagctgccttaact

SEQ ID NO: 26 - DNA, 21 bp, Artificial Sequence
  Synthetic
ggtaacagagctgccttaact

SEQ ID NO: 27 - DNA, 21 bp, Artificial Sequence
  Synthetic
ggaagatgtcaggatggttca

SEQ ID NO: 28 - DNA, 22 bp, Artificial Sequence
  Synthetic
cgaaggacaaattgaggcaaag

SEQ ID NO: 29 - DNA, 21 bp, Artificial Sequence
  Synthetic
tctccttagcagacaattgag

SEQ ID NO: 30 - DNA, 22 bp, Artificial Sequence
  Synthetic
gtgacaaacaagtaggcgaaac

SEQ ID NO: 31 - DNA, 22 bp, Artificial Sequence
  Synthetic
ctaagcaagagcagcaaagaag

SEQ ID NO: 32 - DNA, 22 bp, Artificial Sequence
  Synthetic
catgaactgtggcaacgtaaag

SEQ ID NO: 33 - DNA, 19 bp, Artificial Sequence
  Synthetic
catggccttgtctgtctcc

SEQ ID NO: 34 - DNA, 19 bp, Artificial Sequence
  Synthetic
gaggtatccaaagggcagg

SEQ ID NO: 35 - DNA, 19 bp, Artificial Sequence
  Synthetic
tcatctggctccttgctgg

SEQ ID NO: 36 - DNA, 19 bp, Artificial Sequence
  Synthetic
aacctagcccaaccctcag

SEQ ID NO: 37 - DNA, 22 bp, Artificial Sequence
  Synthetic
cgtcagccagtttgactatgta

SEQ ID NO: 38 - DNA, 22 bp, Artificial Sequence
  Synthetic
ctctcaaagaagctgaggatgg

SEQ ID NO: 39 - DNA, 20 bp, Artificial Sequence
  Synthetic
ccagcaatgccagcatattg

SEQ ID NO: 40 - DNA, 22 bp, Artificial Sequence
  Synthetic
cggtttctccttaccacttctt

SEQ ID NO: 41 - DNA, 22 bp, Artificial Sequence
  Synthetic
gcatccttactctgtggtactg

SEQ ID NO: 42 - DNA, 20 bp, Artificial Sequence
  Synthetic
tatccgcacatcacacatcc

SEQ ID NO: 43 - DNA, 22 bp, Artificial Sequence
  Synthetic
gcaagacgaactaaagcgaaag

SEQ ID NO: 44 - DNA, 20 bp, Artificial Sequence
  Synthetic
tcggaactacgacggtatct

(19) United States
(12) Patent Application Publication
(10) Pub. No.: US 2024/0148671 A1
(43) Pub. Date: May 9, 2024

(54) METHODS FOR IMPROVING POULTRY HEALTH

(71) Applicant: CORNELL UNIVERSITY, Ithaca, NY (US)

(72) Inventor: Elad Tako, Berkshire, NY (US)

(21) Appl. No.: 18/281,440

(22) PCT Filed: Mar. 10, 2022
(86) PCT No.: PCT/US2022/019746

§ 371 Date: Sep. 11, 2023

Related U.S. Application Data
(60) Provisional application No. 63/159,109, filed on Mar. 10, 2021.

(51) Int. Cl. A61K 31/09; A23K 20/111; A23K 50/75; A61K 31/05; A61P 1/00; A61P 3/02; A61P 9/12

(52) U.S. Cl. CPC A61K 31/09; A23K 20/111; A23K 50/75; A61K 31/05; A61P 1/00; A61P 3/02; A61P 9/12

(57) ABSTRACT

Described in certain example embodiments herein are compositions and methods that can, in some embodiments, improve avian health, particularly poultry health. In some embodiments, the method can include administering, to an avian, optionally a poultry animal, an effective amount of a stilbene composition including one or more stilbenes. In some embodiments the stilbene composition including one or more stilbenes includes resveratrol, pterostilbene, piceatannol, or any combination thereof.

### CROSS-REFERENCE TO RELATED APPLICATIONS

This application claims the benefit of and priority to co-pending U.S. Provisional Patent Application No. 63/159,109, filed on Mar. 10, 2021, entitled “METHODS FOR IMPROVING POULTRY HEALTH,” the contents of which is incorporated by reference herein in its entirety.

### SEQUENCE LISTING

This application contains a sequence listing filed in electronic form as an ASCII.txt file entitled CORNL-0800WP ST25.txt, created on Mar. 10, 2022, and having a size of 8,223 bytes. The content of the sequence listing is incorporated herein in its entirety.

### TECHNICAL FIELD

The subject matter disclosed herein is generally directed to compositions and methods for improving poultry health, more particularly methods of administering stilbenes for improving poultry health.

### BACKGROUND

The use of antimicrobials in various livestock industries, including poultry, to prevent and treat disease has been under increasing scrutiny over the past few decades for their role in promoting antimicrobial resistance in key animal and human pathogens. Indeed, over the past several decades poultry producers have faced the implementation of strict government guidelines drastically limiting the use of antimicrobials in production. Several prominent classes of antibiotics, e.g., flouroquinolones and cephalosporins, important in human medicine have been phased out of use in poultry production. Given the continued pressure to reduce the use of antimicrobials for food production, particularly as growth promoters by preventing sub clinical levels of infection, there exists a need for alternatives to antimicrobials for poultry and livestock production.

Citation or identification of any document in this application is not an admission that such a document is available as prior art to the present invention.

### SUMMARY

Described in certain example embodiments herein are methods for improving avian gut health, the method comprising administering, to an avian, optionally a poultry animal, an effective amount of a stilbene composition comprising one or more stilbenes.

In certain example embodiments, the effective amount of the stilbene composition comprises 5-25 mg stilbene/kg of animal body weight.

In certain example embodiments, the stilbene composition comprises resveratrol, pterostilbene, piceatannol, or any combination thereof.

In certain example embodiments, the poultry animal is selected from the group consisting of chicken, turkey, duck, geese, quail, pheasant, emu, and ostrich.

In certain example embodiments, the avian is a wild bird species

In certain example embodiments, the effective amount of the stilbene composition is administered via oral, subcutaneous, intramuscular, or intra-amniotic administration.

In certain example embodiments, the effective amount of the stilbene composition comprises resveratrol and wherein the resveratrol is administered at a concentration of 5-100 mg/ml. In certain example embodiments, effective amount of the stilbene composition comprises pterostilbene and wherein the pterostilbene is administered at a concentration of 5-100 mg/ml. In certain example embodiments, effective amount of the stilbene composition comprises resveratrol and pterostilbene, wherein the resveratrol is administered at a concentration of 2.5-100 mg/ml, and wherein pterostilbene is administered at a concentration of 2.5-100 mg/ml. In certain example embodiments, resveratrol and pterostilbene are administered at a resveratrol to pterostilbene ratio of 95:5.

In certain example embodiments, the effective amount of the stilbene composition is administered via oral administration. In certain example embodiments, the oral administration comprises administering the effective amount of the stilbene composition in feed, in an aqueous solution, or both. In certain example embodiments, the aqueous solution is a water source for the avian.

In certain example embodiments, after administration of the effective amount of the stilbene composition the avian exhibits at least 5% greater villi surface area in the gut after administration; exhibits at least 5% greater goblet cell diameter; exhibits at least 5% increased liver iron concentrations; exhibits at least 5% increased liver zinc concentrations; exhibits at least 5% increased serum iron concentrations; exhibits at least 5% increased serum zinc concentrations; exhibits increased growth rate; exhibits increased bodyweight gain; exhibits decreased feed conversion ratio; comprises a healthy gut microbiome structure; or any combination thereof.

In certain example embodiments, the one or more stilbenes are isolated from grapes, optionally Vitis vinifera, peanuts, pistachios, blueberries, cranberries, tomato, cocoa, hop, rhubarb, strawberry, sugar cane, or any combination thereof.

Described in certain example embodiments herein are avian feed or aqueous formulations comprising an effective amount of a stilbene composition comprising one or more stilbenes, wherein the amount of the stilbene composition is effective to improve avian gut health; and optionally a carrier.

In certain example embodiments, the stilbene composition comprises resveratrol, pterostilbene, piceatannol, or any combination thereof.

In certain example embodiments, the effective amount of the stilbene composition comprises 5-25 mg stilbene/kg of animal body weight.

In certain example embodiments, the effective amount of the stilbene composition comprises resveratrol and wherein the resveratrol is administered at a concentration of 5-100 mg/ml.

In certain example embodiments, the effective amount of the stilbene composition comprises pterostilbene and wherein the pterostilbene is administered at a concentration of 5-100 mg/ml.

In certain example embodiments, the wherein effective amount of the stilbene composition comprises resveratrol and pterostilbene, wherein the resveratrol is administered at a concentration of 2.5-100 mg/ml, and wherein pterostilbene is administered at a concentration of 2.5-100 mg/ml.

In certain example embodiments, resveratrol and pterostilbene are administered at a resveratrol to pterostilbene ratio of 95:5.

In certain example embodiments, the avian feed is adapted for a poultry animal or a wild avian. In certain example embodiments, the poultry animal is selected from the group consisting of chicken, turkey, duck, geese, quail, pheasant, emu, and ostrich.

In certain example embodiments, the avian feed or aqueous formulation is adapted for dilution into a water source. In certain example embodiments, the effective amount of the stilbene composition is effective, in an avian, to increase villi surface area in the gut by at least 5%; increase greater goblet cell diameter by at least 5%; increase liver iron concentrations by at least 5%; increase liver zinc concentration by at least 5%; increase serum iron concentration by at least 5%; increase serum zinc concentration by at least 5%; increase growth rate of an avian; increase bodyweight gain; decrease feed conversion ratio; establish and/or maintain a healthy gut microbiome structure; or any combination thereof.

In certain example embodiments, the one or more stilbenes are isolated from grapes, optionally Vitis vinifera, peanuts, pistachios, blueberries, cranberries, tomato, cocoa, hop, rhubarb, strawberry, sugar cane, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows effect of the intra-amniotic administration of experimental solutions on intestinal gene expression for trace mineral metabolism. Values are the means±SEM, n=8. a-c Per gene, treatments groups not indicated by the same letter are significantly different (p<0.05). DMT1, Divalent metal transporter 1; DcytB, Duodenal cytochrome b; ZnT1, Zinc transporter 1; ZnT7, Zinc transporter 7; ZIP, Zinc transporter 9; TRPV6, Transient Receptor Potential Cation Channel Subfamily V Member 6; PMCA1b, plasma membrane Ca(2+)-ATPase isoform 1b; NCX1, Sodium-calcium exchanger 1; MRS2, Magnesium Transporter; TRPM6, Transient Receptor Potential Cation Channel Subfamily M member 6; TRPM7, Transient Receptor Potential Cation Channel Subfamily M member 7.

FIG. 2 shows effect of the intra-amniotic administration of experimental solutions on intestinal and heart gene expression. Values are the means±SEM, n=8. a—c Per gene, treatments groups not indicated by the same letter are significantly different (p<0.05). IL1β, interleukin 1 (3; IL6, interleukin-6; TNF-α, tumor necrosis factor alpha; AP, Aminopeptidase; SGLT1, Sodium-Glucose transport protein 1; SI, Sucrose isomaltase; ACE, angiotensin-converting enzyme; AT1R, angiotensin II receptor type I.

FIGS. 3A-3C show microbial diversity of the cecal microbiome in the intra-amniotic administration groups. (FIG. 3A) Measure of α-diversity using the Shannon Index; (FIG. 3B) Box-plots of Jaccard similarity differences with significant levels between groups compared to no injection; (FIG. 3C) Measure of β-diversity using Jaccard similarity distances separated by three principal components (PCoA). Each dot represents one animal, and the colors represent the different treatment groups. Values are the means±SEM, n=5, *=p<0.05, **=p<0.01.

FIGS. 4A-4B show compositional changes of gut microbiota in response to the intra-amniotic administration groups. (FIG. 4A) Phylum level changes in the intra-amniotic administration measured at day of hatch (day 21); (FIG. 4B) Genus level changes in the intra-amniotic administration groups as measured on day of hatch (day 21).

FIGS. 5A-5B show LEfSe method identifies the most differentially enriched taxa in the intra-amniotic administration groups. (FIG. 5A) Computed LDA (Linear discriminant analysis) scores of the relative abundance difference between the 5% resveratrol group and the control of no injection. (FIG. 5B) Computer LDA scores of the relative abundance difference between the 5% pterostilbene group and the control of no injection.

FIG. 6A-6D show relative significance of differentially-enriched KEGG microbial metabolic pathways in cecal microbiota. (FIG. 6A) Bacterial Processes; (FIG. 6B) Cellular Processes; (FIG. 6C) Human Disease; FIG. 6D) Metabolic Processes. Treatment groups are indicated by their names and ANOVA values are found within each box.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and

Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

The use of antimicrobials in various livestock industries, including poultry, to prevent and treat disease has been under increasing scrutiny over the past few decades for their role in promoting antimicrobial resistance in key animal and human pathogens. Indeed, over the past several decades poultry producers have faced the implementation of strict government guidelines drastically limiting the use of antimicrobials in production. Several prominent classes of antibiotics, e.g., flouroquinolones and cephalosporins, important in human medicine have been phased out of use in poultry production. Given the continued pressure to reduce the use of antimicrobials for food production, particularly as growth promoters by preventing sub clinical levels of infection, there exists a need for alternatives to antimicrobials for poultry and livestock production.

Polyphenols are a class of bioactive compounds of plant origin that may play an important part in reducing the risk of chronic diseases (Yang et al., Crit. Rev. Food Sci. Nutr. 2013, 53, 1202-1225). Polyphenols are naturally produced by plants as secondary metabolites and are generally involved in plant defense pathways. Recently, there has been growing scientific attention for the health benefits of dietary polyphenols. Epidemiological studies on polyphenol consumption have yielded promising evidence for polyphenols possessing health-promoting properties. These include protection against cardiovascular diseases and diabetes, likely secondary to antioxidant and anti-aging properties (Georgiev et al. Nutrients 2014, 6, 391-415). In a previous epidemiological study that focused on grape-originating polyphenols, researchers devised the expression “the French Paradox” to describe the observed lower coronary heart disease (CHD) mortality in French people despite their high saturated fat intake (Renaud et al. Lancet 1992, 339, 1523-1526). It has been postulated that moderate wine consumption provides a significant amount of dietary polyphenols, thus lowering the incidence of CHD, which may explain this dichotomy.

Resveratrol is a stilbene, a polyphenolic subclass primarily found in grapes (Siemann et al., Am. J. Enol. Vitic. 1992, 43, 49-52). Resveratrol has demonstrated chemoprotective, cardioprotective, anti-inflammatory and neuroprotective activities, likely due to its ability to scavenge free radicals and chelate both redox-active and redox-inactive metals (Berman et al., NPJ Precis. Oncol. 2017, 1, 35 and Cho et al. J. Med. Food 2017, 20, 323-334). Grapes are the most produced fruit crop in the United States. Globally, over 77.8 million tons of grapes are produced annually, with 57% being wine grapes, 36% being table grapes and 7% being dried grapes (International Organization of Vine and Wine. 2019 Statistical Report on World Vitiviniculture; International Organization of Vine and Wine: Paris, France, 2019). Therefore, as grape production and consumption increases, it is imperative to maximize understanding of the specific health effects of bioactive compounds that are found in this fruit. Pterostilbene is another stilbene and an analog of resveratrol present in smaller quantities within grapes and grape products, including wine (Flamini et al., Metabolomics 2013, 9, 1243-1253 and Pezet et al., J. Chromatogr. A 1994, 663, 191-197). The two methoxy substituents in pterostilbene increase the lipophilicity of the compound relative to resveratrol, which contains two hydroxy substituents. In the plant, these compounds serve as the phytoalexins found in grape skins and stems (Bavaresco et al., Drugs Exp. Clin. Res. 1999, 25, 57-63). Pterostilbene is more easily absorbed than resveratrol in the gut lumen because it exhibits higher bioavailability (80%, compared with 20% from resveratrol) (Kapetanovic et al., Cancer Chemother. Pharmacol. 2011, 68, 593-601 and Walle, T. Ann. N. Y. Acad. Sci. 2011, 1215, 9-15).

Although bioactive polyphenols can chelate iron (Fe) and zinc (Zn), studies using various in vitro and in vivo models to evaluate the effects of dietary stilbenes on mineral metabolism are not elucidated (Sreenivasulu et al., J. Food Sci. 2010, 75, H123—H128 and Greger et al., Nutr. 1988, 118, 52-60). This is especially the case with resveratrol and pterostilbene, as there is no substantial research which has evaluated the effects of these compounds on duodenal brush border membrane (BBM) functionality or the gastrointestinal tract microbiota, which play a major role in mineral bioavailability and metabolism (Navarro et al., Compr. Rev. Food Sci. Food Saf. 2018, 17, 808-826). As such it is still unknown if stilbenes can prevent disease and promote growth by improving the overall baseline health of an animal, such as poultry and reduce the need for therapeutic antimicrobial intervention.

With that said, exemplary embodiments disclosed herein can provide are avian feed and aqueous formulations containing an effective amount of a stilbene composition containing one or more stilbenes that, when consumed by the avian, can improve health and/or growth of the avian. Also described in several exemplary embodiments herein are methods of improving avian health by administering an effective amount of a stilbene composition containing one or more stilbenes.” Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Stilbene Compositions and Formulations

Described in certain example embodiments herein are an avian feed or aqueous formulations comprising an effective amount of a stilbene composition comprising one or more stilbenes, wherein the amount of the stilbene composition is effective to improve avian gut health, optionally in a poultry animal and optionally a carrier. In certain example embodiments, the poultry animal is selected from the group consisting of chicken, turkey, duck, geese, quail, pheasant, emu, and ostrich. In certain example embodiments, the avian is a wild bird species. In some embodiments, the wild bird species is an endangered species. Exemplary wild birds include, without limitation, a wild duck, goose, megapode, guan, chachalaca, curassow, wild quail, wild pheasant, grouse, allie, flamingo, grebe, sandgrouse, pigeon, dove, cuckoo, nightjar, potoos, swifts, hummingbird, rail, gallinule, coot, finfoot, limpkin, crane, thick-knees, stilts, avocets, oystercatcher, plover, lapwings, j acanas, sandpiper, pratincoles, courser, skuas, jaeger, auk, murre, puffin, gull, tern, skimmer, tropic bird, loon, albatross, shearwater, petrel, stork , frigatebird, boobie, gannet, anhinga, cormorant, shag, pelican, heron, egret, bitten, ibis, spoonbill, vulture, osprey, hawk, eagle, kite, owl, trogon, hoopoe, todie, kingfisher, roller, woodpecker, falcon, caracara, parrot, cockatoo, tityra, honeyeater, cukooshrike, drongo, fantail, tyrant flycatcher, vireo, shrike-babbler, erporin, monarch flycatcher, shrike, crow, jay, magpie, penduline-tit, tit, chickadee, titmouse, warbler, grassbird, swallow, bulbul, parrotbill, white-eye, yuhina, laughingthrush, kinglet, waxwing, silky-flycatcher, nuthatch, treecreeper, gnatcatcher, wren, thrasher, mockingbird, starling, dipper, thrush, weaver, indigobird, waxbill, accentor, sparrow, wagtail, pipit, finch, euphonia, bunting, longspur, tanager, spindalis, yellow-breasted chat, troupial, cardinal, an others.

In certain example embodiments, the effective amount of the stilbene composition comprises 5-25 mg stilbene(s)/kg of animal body weight. In other words, the total amount of stilbenes can range from 5-25 mg/kg of animal body weight. In some embodiments, the effective amount of the stilbene composition comprises about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg stilbene(s)/kg of animal body weight.

In certain example embodiments, the stilbene composition is contains resveratrol, pterostilbene, piceatannol, or any combination thereof. In certain example embodiments, the stilbene composition is composed only of resveratrol, pterostilbene, piceatannol, or any combination thereof. In some embodiments, the stilbene composition contains or is composed only of resveratrol. In some embodiments, the stilbene composition contains or is composed only of pterostilbene. In some embodiments, the stilbene composition contains or is composed only of piceatannol. In some embodiments, the stilbene composition contains or is composed only of resveratrol and piceatannol. In some embodiments, the stilbene composition contains or is composed only of resveratrol and pterostilbene. In some embodiments, the stilbene composition contains or is composed only of piceatannol and pterostilbene.

The amount of resveratrol, when present in the stilbene composition can range from about 5-25 mg/kg of animal body weight or is present in the stilbene composition at an amount such that when the composition is administered, the resveratrol is administered at an amount ranging from about 5-25 mg/kg of animal bodyweight. In some embodiments, the amount of resveratrol is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

The amount of piceatannol, when present in the stilbene composition can range from about 5-25 mg/kg of animal body weight or is present in the stilbene composition at an amount such that when the composition is administered, the piceatannol is administered at an amount ranging from about 5-25 mg/kg of animal bodyweight. In some embodiments, the amount of piceatannol is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

The amount of pterostilbene, when present in the stilbene composition can range from about 5-25 mg/kg of animal body weight or is present in the stilbene composition at an amount such that when the composition is administered, the pterostilbene is administered at an amount ranging from about 5-25 mg/kg of animal bodyweight. In some embodiments, the amount of pterostilbene is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

In some embodiments, the compositions is a liquid composition and can contain an amount, such as an effective amount, of the stilbene(s) within the liquid composition. In certain example embodiments, the effective amount of the stilbene composition contains or is composed only resveratrol and the resveratrol is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL. In certain example embodiments, effective amount of the stilbene composition contains or is composed only pterostilbene and wherein the pterostilbene is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL. In certain example embodiments, effective amount of the stilbene composition contains or is composed only piceatannol and wherein the piceatannol is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL

In certain example embodiments, the effective amount of the stilbene composition contains or is composed of resveratrol and piceatannol, each of the resveratrol and pterostilbene are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the resveratrol and pterostilbene are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the effective amount of the stilbene composition contains or is composed of resveratrol and piceatannol, each of the resveratrol and piceatannol are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the resveratrol and piceatannol are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the effective amount of the stilbene composition contains or is composed of pterostilbene and piceatannol, each of the pterostilbene and piceatannol are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the pterostilbene and piceatannol are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the piceatannol and pterostilbene are administered at a piceatannol to pterostilbene ratio of 95:5. In certain example embodiments, resveratrol and pterostilbene are administered at a resveratrol to pterostilbene ratio of 95:5. In certain example embodiments, resveratrol and piceatannol are administered at a resveratrol to pterostilbene ratio of 95:5.

The stilbene composition can be or be included in an avian feed, feed supplement, or feed additive that can be consumed by the avian for oral administration. In certain example embodiments, the avian feed, feed supplement, or is adapted for a poultry animal or a wild avian. Exemplary poultry and wild avian are described elsewhere herein. In some embodiments, the amount of the stilbene composition included in a feed additive is such that when added to a feed (such as part of a premix) it is diluted to the effective amount or concentrations described elsewhere herein.

Preparation of feed, feed supplement, and feed additive formulations for avians is generally known in the art. The feed, feed supplement, or feed additive can be prepared in any suitable form, such as a solid, semi-solid, or liquid form. In some embodiments the feed, feed supplement, or feed additive is a pellet, powder, granule, paste, or gel. In some embodiments, the feed is a complete feed. The feed, feed supplement, and/or feed additive can contain one or more binders, colorants, flavors, preservative, and/or the like. Other ingredients of the feed, feed supplement, or feed additive suitable for avians are generally known in the art. In some embodiments, the stilbene composition is integrated with the feed, feed supplement, or feed additive by mixing throughout heterogeneously or homogenously. In some embodiments, the stilbene composition is integrated with the feed, feed supplement, or feed additive by coating it one the outside of the grain, pellet, granule or other feed, feed supplement, or feed additive particle.

In certain example embodiments, the avian feed or aqueous formulation is adapted for delivery by injection. In certain embodiments, the carrier is a liquid suitable for injection via in ovo injection, intramuscular injection, intraamniotic injection, subcutaneous injection, parenteral injection, or other injection.

In certain example embodiments, the avian feed or aqueous formulation is adapted for dilution into a water source such that when the water source is consumed it is ingested by the avian. The amount contained in the liquid formulation, particularly for dilution into a water source, can be an amount that when the water source is consumed at a normal rate by the avian, the amount administered is the effective amount of the stilbene composition, which are described in greater detail elsewhere herein. Water sources include those in poultry houses or other animal enclosures as well as natural sources such as ponds, lakes, streams, etc. In some embodiments, the stilbene composition is formulated for in ovo administration or other injection. Delivery can be manual or automatic, such as via an automatic waterer, feed mixer, in ovo injection machine (e.g., an Embrex machine) and/or the like.

In certain example embodiments, the avian feed or aqueous formulation is adapted for dilution into a water source. In certain example embodiments, the effective amount of the stilbene composition is effective, in an avian, to increase villi surface area in the gut by at least 5%; increase greater goblet cell diameter by at least 5%; increase liver iron concentrations by at least 5%; increase liver zinc concentration by at least 5%; increase serum iron concentration by at least 5%; increase serum zinc concentration by at least 5%; increase growth rate of an avian; increase bodyweight gain; decrease feed conversion ratio; establish and/or maintain a healthy gut microbiome structure; or any combination thereof.

In certain example embodiments, the effective amount of the stilbene composition is effective, in an avian, to increase villi surface area in the gut, such as by 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more. In some embodiments the increase is in the duodenum, jejunum, ileum, or any combination thereof.

In certain example embodiments, the effective amount of the stilbene composition is effective to increase greater goblet cell diameter by 5%-500% or more greater, such as by 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more. In some embodiments the increase is in the duodenum, jejunum, ileum, or any combination thereof.

In certain example embodiments, the effective amount of the stilbene composition is effective to increase liver iron and/or zinc concentration by 5%-500%, such as by 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more.

In certain example embodiments, the effective amount of the stilbene composition increases serum and/or zinc concentration(s) by 5%-500% or more, such as by 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more.

In certain example embodiments, after the effective amount of the stilbene composition increases growth rate, body weight gain, feed efficiency, or any combination thereof in the avian by 0.001%-500% or more, such as by 0.001%, to/or 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, 0.1%, 0.101%, 0.102%, 0.103%, 0.104%, 0.105%, 0.106%, 0.107%, 0.108%, 0.109%, 0.11%, 0.111%, 0.112%, 0.113%, 0.114%, 0.115%, 0.116%, 0.117%, 0.118%, 0.119%, 0.12%, 0.121%, 0.122%, 0.123%, 0.124%, 0.125%, 0.126%, 0.127%, 0.128%, 0.129%, 0.13%, 0.131%, 0.132%, 0.133%, 0.134%, 0.135%, 0.136%, 0.137%, 0.138%, 0.139%, 0.14%, 0.141%, 0.142%, 0.143%, 0.144%, 0.145%, 0.146%, 0.147%, 0.148%, 0.149%, 0.15%, 0.151%, 0.152%, 0.153%, 0.154%, 0.155%, 0.156%, 0.157%, 0.158%, 0.159%, 0.16%, 0.161%, 0.162%, 0.163%, 0.164%, 0.165%, 0.166%, 0.167%, 0.168%, 0.169%, 0.17%, 0.171%, 0.172%, 0.173%, 0.174%, 0.175%, 0.176%, 0.177%, 0.178%, 0.179%, 0.18%, 0.181%, 0.182%, 0.183%, 0.184%, 0.185%, 0.186%, 0.187%, 0.188%, 0.189%, 0.19%, 0.191%, 0.192%, 0.193%, 0.194%, 0.195%, 0.196%, 0.197%, 0.198%, 0.199%, 0.2%, 0.201%, 0.202%, 0.203%, 0.204%, 0.205%, 0.206%, 0.207%, 0.208%, 0.209%, 0.21%, 0.211%, 0.212%, 0.213%, 0.214%, 0.215%, 0.216%, 0.217%, 0.218%, 0.219%, 0.22%, 0.221%, 0.222%, 0.223%, 0.224%, 0.225%, 0.226%, 0.227%, 0.228%, 0.229%, 0.23%, 0.231%, 0.232%, 0.233%, 0.234%, 0.235%, 0.236%, 0.237%, 0.238%, 0.239%, 0.24%, 0.241%, 0.242%, 0.243%, 0.244%, 0.245%, 0.246%, 0.247%, 0.248%, 0.249%, 0.25%, 0.251%, 0.252%, 0.253%, 0.254%, 0.255%, 0.256%, 0.257%, 0.258%, 0.259%, 0.26%, 0.261%, 0.262%, 0.263%, 0.264%, 0.265%, 0.266%, 0.267%, 0.268%, 0.269%, 0.27%, 0.271%, 0.272%, 0.273%, 0.274%, 0.275%, 0.276%, 0.277%, 0.278%, 0.279%, 0.28%, 0.281%, 0.282%, 0.283%, 0.284%, 0.285%, 0.286%, 0.287%, 0.288%, 0.289%, 0.29%, 0.291%, 0.292%, 0.293%, 0.294%, 0.295%, 0.296%, 0.297%, 0.298%, 0.299%, 0.3%, 0.301%, 0.302%, 0.303%, 0.304%, 0.305%, 0.306%, 0.307%, 0.308%, 0.309%, 0.31%, 0.311%, 0.312%, 0.313%, 0.314%, 0.315%, 0.316%, 0.317%, 0.318%, 0.319%, 0.32%, 0.321%, 0.322%, 0.323%, 0.324%, 0.325%, 0.326%, 0.327%, 0.328%, 0.329%, 0.33%, 0.331%, 0.332%, 0.333%, 0.334%, 0.335%, 0.336%, 0.337%, 0.338%, 0.339%, 0.34%, 0.341%, 0.342%, 0.343%, 0.344%, 0.345%, 0.346%, 0.347%, 0.348%, 0.349%, 0.35%, 0.351%, 0.352%, 0.353%, 0.354%, 0.355%, 0.356%, 0.357%, 0.358%, 0.359%, 0.36%, 0.361%, 0.362%, 0.363%, 0.364%, 0.365%, 0.366%, 0.367%, 0.368%, 0.369%, 0.37%, 0.371%, 0.372%, 0.373%, 0.374%, 0.375%, 0.376%, 0.377%, 0.378%, 0.379%, 0.38%, 0.381%, 0.382%, 0.383%, 0.384%, 0.385%, 0.386%, 0.387%, 0.388%, 0.389%, 0.39%, 0.391%, 0.392%, 0.393%, 0.394%, 0.395%, 0.396%, 0.397%, 0.398%, 0.399%, 0.4%, 0.401%, 0.402%, 0.403%, 0.404%, 0.405%, 0.406%, 0.407%, 0.408%, 0.409%, 0.41%, 0.411%, 0.412%, 0.413%, 0.414%, 0.415%, 0.416%, 0.417%, 0.418%, 0.419%, 0.42%, 0.421%, 0.422%, 0.423%, 0.424%, 0.425%, 0.426%, 0.427%, 0.428%, 0.429%, 0.43%, 0.431%, 0.432%, 0.433%, 0.434%, 0.435%, 0.436%, 0.437%, 0.438%, 0.439%, 0.44%, 0.441%, 0.442%, 0.443%, 0.444%, 0.445%, 0.446%, 0.447%, 0.448%, 0.449%, 0.45%, 0.451%, 0.452%, 0.453%, 0.454%, 0.455%, 0.456%, 0.457%, 0.458%, 0.459%, 0.46%, 0.461%, 0.462%, 0.463%, 0.464%, 0.465%, 0.466%, 0.467%, 0.468%, 0.469%, 0.47%, 0.471%, 0.472%, 0.473%, 0.474%, 0.475%, 0.476%, 0.477%, 0.478%, 0.479%, 0.48%, 0.481%, 0.482%, 0.483%, 0.484%, 0.485%, 0.486%, 0.487%, 0.488%, 0.489%, 0.49%, 0.491%, 0.492%, 0.493%, 0.494%, 0.495%, 0.496%, 0.497%, 0.498%, 0.499%, 0.5%, 0.501%, 0.502%, 0.503%, 0.504%, 0.505%, 0.506%, 0.507%, 0.508%, 0.509%, 0.51%, 0.511%, 0.512%, 0.513%, 0.514%, 0.515%, 0.516%, 0.517%, 0.518%, 0.519%, 0.52%, 0.521%, 0.522%, 0.523%, 0.524%, 0.525%, 0.526%, 0.527%, 0.528%, 0.529%, 0.53%, 0.531%, 0.532%, 0.533%, 0.534%, 0.535%, 0.536%, 0.537%, 0.538%, 0.539%, 0.54%, 0.541%, 0.542%, 0.543%, 0.544%, 0.545%, 0.546%, 0.547%, 0.548%, 0.549%, 0.55%, 0.551%, 0.552%, 0.553%, 0.554%, 0.555%, 0.556%, 0.557%, 0.558%, 0.559%, 0.56%, 0.561%, 0.562%, 0.563%, 0.564%, 0.565%, 0.566%, 0.567%, 0.568%, 0.569%, 0.57%, 0.571%, 0.572%, 0.573%, 0.574%, 0.575%, 0.576%, 0.577%, 0.578%, 0.579%, 0.58%, 0.581%, 0.582%, 0.583%, 0.584%, 0.585%, 0.586%, 0.587%, 0.588%, 0.589%, 0.59%, 0.591%, 0.592%, 0.593%, 0.594%, 0.595%, 0.596%, 0.597%, 0.598%, 0.599%, 0.6%, 0.601%, 0.602%, 0.603%, 0.604%, 0.605%, 0.606%, 0.607%, 0.608%, 0.609%, 0.61%, 0.611%, 0.612%, 0.613%, 0.614%, 0.615%, 0.616%, 0.617%, 0.618%, 0.619%, 0.62%, 0.621%, 0.622%, 0.623%, 0.624%, 0.625%, 0.626%, 0.627%, 0.628%, 0.629%, 0.63%, 0.631%, 0.632%, 0.633%, 0.634%, 0.635%, 0.636%, 0.637%, 0.638%, 0.639%, 0.64%, 0.641%, 0.642%, 0.643%, 0.644%, 0.645%, 0.646%, 0.647%, 0.648%, 0.649%, 0.65%, 0.651%, 0.652%, 0.653%, 0.654%, 0.655%, 0.656%, 0.657%, 0.658%, 0.659%, 0.66%, 0.661%, 0.662%, 0.663%, 0.664%, 0.665%, 0.666%, 0.667%, 0.668%, 0.669%, 0.67%, 0.671%, 0.672%, 0.673%, 0.674%, 0.675%, 0.676%, 0.677%, 0.678%, 0.679%, 0.68%, 0.681%, 0.682%, 0.683%, 0.684%, 0.685%, 0.686%, 0.687%, 0.688%, 0.689%, 0.69%, 0.691%, 0.692%, 0.693%, 0.694%, 0.695%, 0.696%, 0.697%, 0.698%, 0.699%, 0.7%, 0.701%, 0.702%, 0.703%, 0.704%, 0.705%, 0.706%, 0.707%, 0.708%, 0.709%, 0.71%, 0.711%, 0.712%, 0.713%, 0.714%, 0.715%, 0.716%, 0.717%, 0.718%, 0.719%, 0.72%, 0.721%, 0.722%, 0.723%, 0.724%, 0.725%, 0.726%, 0.727%, 0.728%, 0.729%, 0.73%, 0.731%, 0.732%, 0.733%, 0.734%, 0.735%, 0.736%, 0.737%, 0.738%, 0.739%, 0.74%, 0.741%, 0.742%, 0.743%, 0.744%, 0.745%, 0.746%, 0.747%, 0.748%, 0.749%, 0.75%, 0.751%, 0.752%, 0.753%, 0.754%, 0.755%, 0.756%, 0.757%, 0.758%, 0.759%, 0.76%, 0.761%, 0.762%, 0.763%, 0.764%, 0.765%, 0.766%, 0.767%, 0.768%, 0.769%, 0.77%, 0.771%, 0.772%, 0.773%, 0.774%, 0.775%, 0.776%, 0.777%, 0.778%, 0.779%, 0.78%, 0.781%, 0.782%, 0.783%, 0.784%, 0.785%, 0.786%, 0.787%, 0.788%, 0.789%, 0.79%, 0.791%, 0.792%, 0.793%, 0.794%, 0.795%, 0.796%, 0.797%, 0.798%, 0.799%, 0.8%, 0.801%, 0.802%, 0.803%, 0.804%, 0.805%, 0.806%, 0.807%, 0.808%, 0.809%, 0.81%, 0.811%, 0.812%, 0.813%, 0.814%, 0.815%, 0.816%, 0.817%, 0.818%, 0.819%, 0.82%, 0.821%, 0.822%, 0.823%, 0.824%, 0.825%, 0.826%, 0.827%, 0.828%, 0.829%, 0.83%, 0.831%, 0.832%, 0.833%, 0.834%, 0.835%, 0.836%, 0.837%, 0.838%, 0.839%, 0.84%, 0.841%, 0.842%, 0.843%, 0.844%, 0.845%, 0.846%, 0.847%, 0.848%, 0.849%, 0.85%, 0.851%, 0.852%, 0.853%, 0.854%, 0.855%, 0.856%, 0.857%, 0.858%, 0.859%, 0.86%, 0.861%, 0.862%, 0.863%, 0.864%, 0.865%, 0.866%, 0.867%, 0.868%, 0.869%, 0.87%, 0.871%, 0.872%, 0.873%, 0.874%, 0.875%, 0.876%, 0.877%, 0.878%, 0.879%, 0.88%, 0.881%, 0.882%, 0.883%, 0.884%, 0.885%, 0.886%, 0.887%, 0.888%, 0.889%, 0.89%, 0.891%, 0.892%, 0.893%, 0.894%, 0.895%, 0.896%, 0.897%, 0.898%, 0.899%, 0.9%, 0.901%, 0.902%, 0.903%, 0.904%, 0.905%, 0.906%, 0.907%, 0.908%, 0.909%, 0.91%, 0.911%, 0.912%, 0.913%, 0.914%, 0.915%, 0.916%, 0.917%, 0.918%, 0.919%, 0.92%, 0.921%, 0.922%, 0.923%, 0.924%, 0.925%, 0.926%, 0.927%, 0.928%, 0.929%, 0.93%, 0.931%, 0.932%, 0.933%, 0.934%, 0.935%, 0.936%, 0.937%, 0.938%, 0.939%, 0.94%, 0.941%, 0.942%, 0.943%, 0.944%, 0.945%, 0.946%, 0.947%, 0.948%, 0.949%, 0.95%, 0.951%, 0.952%, 0.953%, 0.954%, 0.955%, 0.956%, 0.957%, 0.958%, 0.959%, 0.96%, 0.961%, 0.962%, 0.963%, 0.964%, 0.965%, 0.966%, 0.967%, 0.968%, 0.969%, 0.97%, 0.971%, 0.972%, 0.973%, 0.974%, 0.975%, 0.976%, 0.977%, 0.978%, 0.979%, 0.98%, 0.981%, 0.982%, 0.983%, 0.984%, 0.985%, 0.986%, 0.987%, 0.988%, 0.989%, 0.99%, 0.991%, 0.992%, 0.993%, 0.994%, 0.995%, 0.996%, 0.997%, 0.998%, 0.999%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more.

In certain example embodiments, the effective amount of the stilbene composition decreases feed conversion ratio by 0.001%-1% or more such as by 0.001%, to/or 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, 0.1%, 0.101%, 0.102%, 0.103%, 0.104%, 0.105%, 0.106%, 0.107%, 0.108%, 0.109%, 0.11%, 0.111%, 0.112%, 0.113%, 0.114%, 0.115%, 0.116%, 0.117%, 0.118%, 0.119%, 0.12%, 0.121%, 0.122%, 0.123%, 0.124%, 0.125%, 0.126%, 0.127%, 0.128%, 0.129%, 0.13%, 0.131%, 0.132%, 0.133%, 0.134%, 0.135%, 0.136%, 0.137%, 0.138%, 0.139%, 0.14%, 0.141%, 0.142%, 0.143%, 0.144%, 0.145%, 0.146%, 0.147%, 0.148%, 0.149%, 0.15%, 0.151%, 0.152%, 0.153%, 0.154%, 0.155%, 0.156%, 0.157%, 0.158%, 0.159%, 0.16%, 0.161%, 0.162%, 0.163%, 0.164%, 0.165%, 0.166%, 0.167%, 0.168%, 0.169%, 0.17%, 0.171%, 0.172%, 0.173%, 0.174%, 0.175%, 0.176%, 0.177%, 0.178%, 0.179%, 0.18%, 0.181%, 0.182%, 0.183%, 0.184%, 0.185%, 0.186%, 0.187%, 0.188%, 0.189%, 0.19%, 0.191%, 0.192%, 0.193%, 0.194%, 0.195%, 0.196%, 0.197%, 0.198%, 0.199%, 0.2%, 0.201%, 0.202%, 0.203%, 0.204%, 0.205%, 0.206%, 0.207%, 0.208%, 0.209%, 0.21%, 0.211%, 0.212%, 0.213%, 0.214%, 0.215%, 0.216%, 0.217%, 0.218%, 0.219%, 0.22%, 0.221%, 0.222%, 0.223%, 0.224%, 0.225%, 0.226%, 0.227%, 0.228%, 0.229%, 0.23%, 0.231%, 0.232%, 0.233%, 0.234%, 0.235%, 0.236%, 0.237%, 0.238%, 0.239%, 0.24%, 0.241%, 0.242%, 0.243%, 0.244%, 0.245%, 0.246%, 0.247%, 0.248%, 0.249%, 0.25%, 0.251%, 0.252%, 0.253%, 0.254%, 0.255%, 0.256%, 0.257%, 0.258%, 0.259%, 0.26%, 0.261%, 0.262%, 0.263%, 0.264%, 0.265%, 0.266%, 0.267%, 0.268%, 0.269%, 0.27%, 0.271%, 0.272%, 0.273%, 0.274%, 0.275%, 0.276%, 0.277%, 0.278%, 0.279%, 0.28%, 0.281%, 0.282%, 0.283%, 0.284%, 0.285%, 0.286%, 0.287%, 0.288%, 0.289%, 0.29%, 0.291%, 0.292%, 0.293%, 0.294%, 0.295%, 0.296%, 0.297%, 0.298%, 0.299%, 0.3%, 0.301%, 0.302%, 0.303%, 0.304%, 0.305%, 0.306%, 0.307%, 0.308%, 0.309%, 0.31%, 0.311%, 0.312%, 0.313%, 0.314%, 0.315%, 0.316%, 0.317%, 0.318%, 0.319%, 0.32%, 0.321%, 0.322%, 0.323%, 0.324%, 0.325%, 0.326%, 0.327%, 0.328%, 0.329%, 0.33%, 0.331%, 0.332%, 0.333%, 0.334%, 0.335%, 0.336%, 0.337%, 0.338%, 0.339%, 0.34%, 0.341%, 0.342%, 0.343%, 0.344%, 0.345%, 0.346%, 0.347%, 0.348%, 0.349%, 0.35%, 0.351%, 0.352%, 0.353%, 0.354%, 0.355%, 0.356%, 0.357%, 0.358%, 0.359%, 0.36%, 0.361%, 0.362%, 0.363%, 0.364%, 0.365%, 0.366%, 0.367%, 0.368%, 0.369%, 0.37%, 0.371%, 0.372%, 0.373%, 0.374%, 0.375%, 0.376%, 0.377%, 0.378%, 0.379%, 0.38%, 0.381%, 0.382%, 0.383%, 0.384%, 0.385%, 0.386%, 0.387%, 0.388%, 0.389%, 0.39%, 0.391%, 0.392%, 0.393%, 0.394%, 0.395%, 0.396%, 0.397%, 0.398%, 0.399%, 0.4%, 0.401%, 0.402%, 0.403%, 0.404%, 0.405%, 0.406%, 0.407%, 0.408%, 0.409%, 0.41%, 0.411%, 0.412%, 0.413%, 0.414%, 0.415%, 0.416%, 0.417%, 0.418%, 0.419%, 0.42%, 0.421%, 0.422%, 0.423%, 0.424%, 0.425%, 0.426%, 0.427%, 0.428%, 0.429%, 0.43%, 0.431%, 0.432%, 0.433%, 0.434%, 0.435%, 0.436%, 0.437%, 0.438%, 0.439%, 0.44%, 0.441%, 0.442%, 0.443%, 0.444%, 0.445%, 0.446%, 0.447%, 0.448%, 0.449%, 0.45%, 0.451%, 0.452%, 0.453%, 0.454%, 0.455%, 0.456%, 0.457%, 0.458%, 0.459%, 0.46%, 0.461%, 0.462%, 0.463%, 0.464%, 0.465%, 0.466%, 0.467%, 0.468%, 0.469%, 0.47%, 0.471%, 0.472%, 0.473%, 0.474%, 0.475%, 0.476%, 0.477%, 0.478%, 0.479%, 0.48%, 0.481%, 0.482%, 0.483%, 0.484%, 0.485%, 0.486%, 0.487%, 0.488%, 0.489%, 0.49%, 0.491%, 0.492%, 0.493%, 0.494%, 0.495%, 0.496%, 0.497%, 0.498%, 0.499%, 0.5%, 0.501%, 0.502%, 0.503%, 0.504%, 0.505%, 0.506%, 0.507%, 0.508%, 0.509%, 0.51%, 0.511%, 0.512%, 0.513%, 0.514%, 0.515%, 0.516%, 0.517%, 0.518%, 0.519%, 0.52%, 0.521%, 0.522%, 0.523%, 0.524%, 0.525%, 0.526%, 0.527%, 0.528%, 0.529%, 0.53%, 0.531%, 0.532%, 0.533%, 0.534%, 0.535%, 0.536%, 0.537%, 0.538%, 0.539%, 0.54%, 0.541%, 0.542%, 0.543%, 0.544%, 0.545%, 0.546%, 0.547%, 0.548%, 0.549%, 0.55%, 0.551%, 0.552%, 0.553%, 0.554%, 0.555%, 0.556%, 0.557%, 0.558%, 0.559%, 0.56%, 0.561%, 0.562%, 0.563%, 0.564%, 0.565%, 0.566%, 0.567%, 0.568%, 0.569%, 0.57%, 0.571%, 0.572%, 0.573%, 0.574%, 0.575%, 0.576%, 0.577%, 0.578%, 0.579%, 0.58%, 0.581%, 0.582%, 0.583%, 0.584%, 0.585%, 0.586%, 0.587%, 0.588%, 0.589%, 0.59%, 0.591%, 0.592%, 0.593%, 0.594%, 0.595%, 0.596%, 0.597%, 0.598%, 0.599%, 0.6%, 0.601%, 0.602%, 0.603%, 0.604%, 0.605%, 0.606%, 0.607%, 0.608%, 0.609%, 0.61%, 0.611%, 0.612%, 0.613%, 0.614%, 0.615%, 0.616%, 0.617%, 0.618%, 0.619%, 0.62%, 0.621%, 0.622%, 0.623%, 0.624%, 0.625%, 0.626%, 0.627%, 0.628%, 0.629%, 0.63%, 0.631%, 0.632%, 0.633%, 0.634%, 0.635%, 0.636%, 0.637%, 0.638%, 0.639%, 0.64%, 0.641%, 0.642%, 0.643%, 0.644%, 0.645%, 0.646%, 0.647%, 0.648%, 0.649%, 0.65%, 0.651%, 0.652%, 0.653%, 0.654%, 0.655%, 0.656%, 0.657%, 0.658%, 0.659%, 0.66%, 0.661%, 0.662%, 0.663%, 0.664%, 0.665%, 0.666%, 0.667%, 0.668%, 0.669%, 0.67%, 0.671%, 0.672%, 0.673%, 0.674%, 0.675%, 0.676%, 0.677%, 0.678%, 0.679%, 0.68%, 0.681%, 0.682%, 0.683%, 0.684%, 0.685%, 0.686%, 0.687%, 0.688%, 0.689%, 0.69%, 0.691%, 0.692%, 0.693%, 0.694%, 0.695%, 0.696%, 0.697%, 0.698%, 0.699%, 0.7%, 0.701%, 0.702%, 0.703%, 0.704%, 0.705%, 0.706%, 0.707%, 0.708%, 0.709%, 0.71%, 0.711%, 0.712%, 0.713%, 0.714%, 0.715%, 0.716%, 0.717%, 0.718%, 0.719%, 0.72%, 0.721%, 0.722%, 0.723%, 0.724%, 0.725%, 0.726%, 0.727%, 0.728%, 0.729%, 0.73%, 0.731%, 0.732%, 0.733%, 0.734%, 0.735%, 0.736%, 0.737%, 0.738%, 0.739%, 0.74%, 0.741%, 0.742%, 0.743%, 0.744%, 0.745%, 0.746%, 0.747%, 0.748%, 0.749%, 0.75%, 0.751%, 0.752%, 0.753%, 0.754%, 0.755%, 0.756%, 0.757%, 0.758%, 0.759%, 0.76%, 0.761%, 0.762%, 0.763%, 0.764%, 0.765%, 0.766%, 0.767%, 0.768%, 0.769%, 0.77%, 0.771%, 0.772%, 0.773%, 0.774%, 0.775%, 0.776%, 0.777%, 0.778%, 0.779%, 0.78%, 0.781%, 0.782%, 0.783%, 0.784%, 0.785%, 0.786%, 0.787%, 0.788%, 0.789%, 0.79%, 0.791%, 0.792%, 0.793%, 0.794%, 0.795%, 0.796%, 0.797%, 0.798%, 0.799%, 0.8%, 0.801%, 0.802%, 0.803%, 0.804%, 0.805%, 0.806%, 0.807%, 0.808%, 0.809%, 0.81%, 0.811%, 0.812%, 0.813%, 0.814%, 0.815%, 0.816%, 0.817%, 0.818%, 0.819%, 0.82%, 0.821%, 0.822%, 0.823%, 0.824%, 0.825%, 0.826%, 0.827%, 0.828%, 0.829%, 0.83%, 0.831%, 0.832%, 0.833%, 0.834%, 0.835%, 0.836%, 0.837%, 0.838%, 0.839%, 0.84%, 0.841%, 0.842%, 0.843%, 0.844%, 0.845%, 0.846%, 0.847%, 0.848%, 0.849%, 0.85%, 0.851%, 0.852%, 0.853%, 0.854%, 0.855%, 0.856%, 0.857%, 0.858%, 0.859%, 0.86%, 0.861%, 0.862%, 0.863%, 0.864%, 0.865%, 0.866%, 0.867%, 0.868%, 0.869%, 0.87%, 0.871%, 0.872%, 0.873%, 0.874%, 0.875%, 0.876%, 0.877%, 0.878%, 0.879%, 0.88%, 0.881%, 0.882%, 0.883%, 0.884%, 0.885%, 0.886%, 0.887%, 0.888%, 0.889%, 0.89%, 0.891%, 0.892%, 0.893%, 0.894%, 0.895%, 0.896%, 0.897%, 0.898%, 0.899%, 0.9%, 0.901%, 0.902%, 0.903%, 0.904%, 0.905%, 0.906%, 0.907%, 0.908%, 0.909%, 0.91%, 0.911%, 0.912%, 0.913%, 0.914%, 0.915%, 0.916%, 0.917%, 0.918%, 0.919%, 0.92%, 0.921%, 0.922%, 0.923%, 0.924%, 0.925%, 0.926%, 0.927%, 0.928%, 0.929%, 0.93%, 0.931%, 0.932%, 0.933%, 0.934%, 0.935%, 0.936%, 0.937%, 0.938%, 0.939%, 0.94%, 0.941%, 0.942%, 0.943%, 0.944%, 0.945%, 0.946%, 0.947%, 0.948%, 0.949%, 0.95%, 0.951%, 0.952%, 0.953%, 0.954%, 0.955%, 0.956%, 0.957%, 0.958%, 0.959%, 0.96%, 0.961%, 0.962%, 0.963%, 0.964%, 0.965%, 0.966%, 0.967%, 0.968%, 0.969%, 0.97%, 0.971%, 0.972%, 0.973%, 0.974%, 0.975%, 0.976%, 0.977%, 0.978%, 0.979%, 0.98%, 0.981%, 0.982%, 0.983%, 0.984%, 0.985%, 0.986%, 0.987%, 0.988%, 0.989%, 0.99%, 0.991%, 0.992%, 0.993%, 0.994%, 0.995%, 0.996%, 0.997%, 0.998%, 0.999%, or 1% or more.

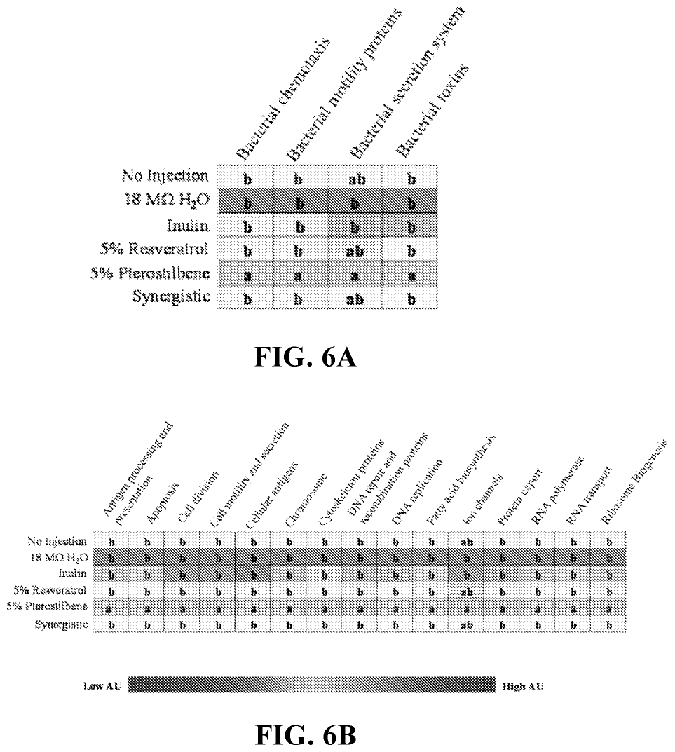

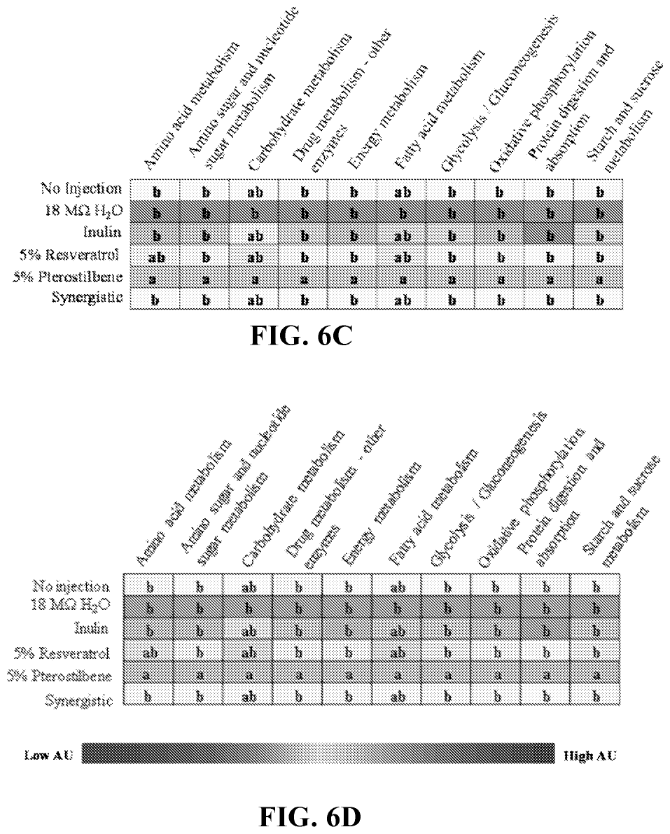

In some embodiments, the effective amount of the stilbene composition can improve the health of a gut microbiome and/or promote a healthy microbiome structure. As used herein “microbiome structure” refers to the profile of microbial species and subspecies present in the microbiome. In some embodiments, the effective amount of the stilbene composition increases the number of non-pathogenic and/or beneficial bacteria and decreases the number of pathogenic bacteria thus promoting a healthy microbiome structure. In some embodiments, the health and/or functionality of the microbes in the microbiome are improved. For example, the effective amount of the stilbene composition can improve cellular processes like DNA repair and recombination proteins and fatty acid biosynthesis, as well as improved metabolic processes like carbohydrate metabolism and energy metabolism relative to the naive system (no-injection control) (see e.g., FIG. 6A-6D).

In certain example embodiments, the one or more stilbenes are isolated from grapes, optionally Vitis vinifera, peanuts, pistachios, blueberries, cranberries, tomato, cocoa, hop, rhubarb, strawberry, sugar cane, or any combination thereof.

### Methods of Use

Described in certain example embodiments herein are methods for improving avian gut health, optionally poultry gut health, the method comprising administering, to an avian, optionally a poultry animal, an effective amount of a stilbene composition comprising one or more stilbenes. Without being bound by theory, improving gut health can result in performance gains (e.g., increased body weight gain, feed efficiency, and/or the like. It is believed that the compositions herein can promote improved nutrient assimilation of key nutrients such as iron and zinc. Further, improvement in the overall gut health may improve disease resistance and/or tolerance and thus reduce the need for antimicrobials. As is also demonstrated in the Working Examples, the methods provided herein can promote a healthy microbiome structure. Without being bound by theory, a healthy gut microbiome can prevent colonization of pathogenic bacteria in the gut and thus improve health and promote disease resistance. Further a healthy gut microbiome can have improved functionality that can produce beneficial products, such as fatty acids, and others that can be used by the animal. In certain example embodiments, the one or more stilbenes are isolated from grapes, optionally Vitis vinifera, peanuts, pistachios, blueberries, cranberries, tomato, cocoa, hop, rhubarb, strawberry, sugar cane, or any combination thereof.

In certain example embodiments, the effective amount of the stilbene composition comprises 5-25 mg stilbene(s)/kg of animal body weight. In other words, the total amount of stilbenes can range from 5-25 mg/kg of animal body weight. In some embodiments, the effective amount of the stilbene composition comprises about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg stilbene(s)/kg of animal body weight.

In certain example embodiments, the stilbene composition is contains resveratrol, pterostilbene, piceatannol, or any combination thereof. In certain example embodiments, the stilbene composition is composed only of resveratrol, pterostilbene, piceatannol, or any combination thereof. In some embodiments, the stilbene composition contains or is composed only of resveratrol. In some embodiments, the stilbene composition contains or is composed only of pterostilbene. In some embodiments, the stilbene composition contains or is composed only of piceatannol. In some embodiments, the stilbene composition contains or is composed only of resveratrol and piceatannol. In some embodiments, the stilbene composition contains or is composed only of resveratrol and pterostilbene. In some embodiments, the stilbene composition contains or is composed only of piceatannol and pterostilbene.

The amount of resveratrol, when present in the stilbene composition can range from about 5-25 mg/kg of animal body weight or is present in the stilbene composition at an amount such that when the composition is administered, the resveratrol is administered at an amount ranging from about 5-25 mg/kg of animal bodyweight. In some embodiments, the amount of resveratrol is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

The amount of piceatannol, when present in the stilbene composition can range from about 5-25 mg/kg of animal body weight or is present in the stilbene composition at an amount such that when the composition is administered, the piceatannol is administered at an amount ranging from about 5-25 mg/kg of animal bodyweight. In some embodiments, the amount of piceatannol is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

The amount of pterostilbene, when present in the stilbene composition can range from about 5-25 mg/kg of animal body weight or is present in the stilbene composition at an amount such that when the composition is administered, the pterostilbene is administered at an amount ranging from about 5-25 mg/kg of animal bodyweight. In some embodiments, the amount of pterostilbene is or is administered at about 5, to/or 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or 25 mg/kg of animal body weight.

In some embodiments, the compositions is a liquid composition and can contain an amount, such as an effective amount, of the stilbene(s) within the liquid composition. In certain example embodiments, the effective amount of the stilbene composition contains or is composed only resveratrol and the resveratrol is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL. In certain example embodiments, effective amount of the stilbene composition contains or is composed only pterostilbene and wherein the pterostilbene is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL. In certain example embodiments, effective amount of the stilbene composition contains or is composed only piceatannol and wherein the piceatannol is administered at a concentration of 5-100 mg/ml, such as 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL.

In certain example embodiments, the effective amount of the stilbene composition contains or is composed of resveratrol and piceatannol, each of the resveratrol and pterostilbene are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the resveratrol and pterostilbene are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the effective amount of the stilbene composition contains or is composed of resveratrol and piceatannol, each of the resveratrol and piceatannol are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the resveratrol and piceatannol are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the effective amount of the stilbene composition contains or is composed of pterostilbene and piceatannol, each of the pterostilbene and piceatannol are present are concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL) or are each present at an amount such that, when administered, the each of the pterostilbene and piceatannol are administered at a concentration ranging from 2.5-100 mg/mL (e.g., 2.5, to/or 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 mg/mL).

In certain example embodiments, the piceatannol and pterostilbene are administered at a piceatannol to pterostilbene ratio of 95:5. In certain example embodiments, resveratrol and pterostilbene are administered at a resveratrol to pterostilbene ratio of 95:5. In certain example embodiments, resveratrol and piceatannol are administered at a resveratrol to pterostilbene ratio of 95:5.

In certain example embodiments, the poultry animal is selected from the group consisting of chicken, turkey, duck, geese, quail, pheasant, emu, and ostrich.

In certain example embodiments, the avian is a wild bird species. In some embodiments, the wild bird species is an endangered species. Exemplary wild birds include, without limitation, a wild duck, goose, megapode, guan, chachalaca, curassow, wild quail, wild pheasant, grouse, allie, flamingo, grebe, sandgrouse, pigeon, dove, cuckoo, nightjar, potoos, swifts, hummingbird, rail, gallinule, coot, finfoot, limpkin, crane, thick-knees, stilts, avocets, oystercatcher, plover, lapwings, jacanas, sandpiper, pratincoles, courser, skuas, jaeger, auk, murre, puffin, gull, tern, skimmer, tropic bird, loon, albatross, shearwater, petrel, stork , frigatebird, boobie, gannet, anhinga, cormorant, shag, pelican, heron, egret, bitten, ibis, spoonbill, vulture, osprey, hawk, eagle, kite, owl, trogon, hoopoe, todie, kingfisher, roller, woodpecker, falcon, caracara, parrot, cockatoo, tityra, honeyeater, cukooshrike, drongo, fantail, tyrant flycatcher, vireo, shrike-babbler, erporin, monarch flycatcher, shrike, crow, jay, magpie, penduline-tit, tit, chickadee, titmouse, warbler, grassbird, swallow, bulbul, parrotbill, white-eye, yuhina, laughingthrush, kinglet, waxwing, silky-flycatcher, nuthatch, treecreeper, gnatcatcher, wren, thrasher, mockingbird, starling, dipper, thrush, weaver, indigobird, waxbill, accentor, sparrow, wagtail, pipit, finch, euphonia, bunting, longspur, tanager, spindalis, yellow-breasted chat, troupial, cardinal, an others.

The stilbene composition can be administered by any suitable route. In certain example embodiments, the effective amount of the stilbene composition is administered via oral, subcutaneous, intramuscular, or intra-amniotic administration. The stilbene composition can be administered by any suitable route. In certain example embodiments, the effective amount of the stilbene composition is administered via oral administration. In certain example embodiments, the oral administration comprises administering the effective amount of the stilbene composition in feed, in an aqueous or other liquid solution, or both. In certain example embodiments, the aqueous or other liquid solution is a water source for the avian.

In some embodiments, it is mixed with, coated on, or otherwise integrated with a feed to feed the avian for oral administration. In some embodiments, it can be formulated in a liquid formulation that can be added to a water source such that when the water source is consumed it is ingested by the avian. Water sources include those in poultry houses or other animal enclosures as well as natural sources such as ponds, lakes, streams, etc. In some embodiments, the stilbene composition is formulated for in ovo administration or other injection. Delivery can be manual or automatic, such as via an automatic waterer, feed mixer, in ovo injection machine (e.g., an Embrex machine) and/or the like.

Administration can be daily, every-other day, every third day, every fourth day, every fifth day, every sixth day, weekly, or monthly. In some embodiments, administration is a single administration.

After administration, the avian can have one or more improvements in performance and/or health, particularly gut health. There are many performance parameters that can be measured to determine if performance is improved. Such parameters will be appreciated by those of ordinary skill in the art and include, without limitation, growth rate, body weight, body weight gain, feed conversion, etc. Such parameters can also be indicative of overall improved health. Without being bound by theory improving the health, particularly gut health and function, can result in performance benefits.

In certain example embodiments, after administration of the effective amount of the stilbene composition the avian exhibits at least 5% greater villi surface area in the gut; exhibits at least 5% greater goblet cell diameter; exhibits at least 5% increased liver iron concentrations; exhibits at least 5% increased liver zinc concentrations; exhibits at least 5% increased serum iron concentrations; exhibits at least 5% increased serum zinc concentrations; exhibits increased growth rate; exhibits increased bodyweight gain; exhibits decreased feed conversion ratio; comprises a healthy gut microbiome structure; or any combination thereof.

In certain example embodiments, after administration of the effective amount of the stilbene composition the avian exhibits 5%-500% or more greater villi surface area in the gut, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more greater surface area villi surface area in the gut. In some embodiments the increase is in the duodenum, jejunum, ileum, or any combination thereof.

In certain example embodiments, after administration of the effective amount of the stilbene composition the avian exhibits 5%-500% or more greater goblet cell diameter, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more greater goblet cell diameter. In some embodiments the increase is in the duodenum, jejunum, ileum, or any combination thereof.

In certain example embodiments, after administration of the effective amount of the stilbene composition the avian exhibits 5%-500% or more increased liver iron concentration, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more increased liver iron concentration.

In certain example embodiments, after administration of the effective amount of the stilbene composition the avian exhibits 5%-500% or more increased liver zinc concentration, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more increased liver zinc concentration.

In certain example embodiments, after administration of the effective amount of the stilbene composition the avian exhibits 5%-500% or more increased serum iron concentration, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more increased serum iron concentration.

In certain example embodiments, after administration of the effective amount of the stilbene composition the avian exhibits 5%-500% or more increased serum zinc concentration, such as 5%, to/or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more increased serum zinc concentration.

In certain example embodiments, after administration of the effective amount of the stilbene composition the avian exhibits 0.001%-500% or more increase in growth rate, body weight gain, feed efficiency, or any combination thereof, such as 0.001%, to/or 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, 0.1%, 0.101%, 0.102%, 0.103%, 0.104%, 0.105%, 0.106%, 0.107%, 0.108%, 0.109%, 0.11%, 0.111%, 0.112%, 0.113%, 0.114%, 0.115%, 0.116%, 0.117%, 0.118%, 0.119%, 0.12%, 0.121%, 0.122%, 0.123%, 0.124%, 0.125%, 0.126%, 0.127%, 0.128%, 0.129%, 0.13%, 0.131%, 0.132%, 0.133%, 0.134%, 0.135%, 0.136%, 0.137%, 0.138%, 0.139%, 0.14%, 0.141%, 0.142%, 0.143%, 0.144%, 0.145%, 0.146%, 0.147%, 0.148%, 0.149%, 0.15%, 0.151%, 0.152%, 0.153%, 0.154%, 0.155%, 0.156%, 0.157%, 0.158%, 0.159%, 0.16%, 0.161%, 0.162%, 0.163%, 0.164%, 0.165%, 0.166%, 0.167%, 0.168%, 0.169%, 0.17%, 0.171%, 0.172%, 0.173%, 0.174%, 0.175%, 0.176%, 0.177%, 0.178%, 0.179%, 0.18%, 0.181%, 0.182%, 0.183%, 0.184%, 0.185%, 0.186%, 0.187%, 0.188%, 0.189%, 0.19%, 0.191%, 0.192%, 0.193%, 0.194%, 0.195%, 0.196%, 0.197%, 0.198%, 0.199%, 0.2%, 0.201%, 0.202%, 0.203%, 0.204%, 0.205%, 0.206%, 0.207%, 0.208%, 0.209%, 0.21%, 0.211%, 0.212%, 0.213%, 0.214%, 0.215%, 0.216%, 0.217%, 0.218%, 0.219%, 0.22%, 0.221%, 0.222%, 0.223%, 0.224%, 0.225%, 0.226%, 0.227%, 0.228%, 0.229%, 0.23%, 0.231%, 0.232%, 0.233%, 0.234%, 0.235%, 0.236%, 0.237%, 0.238%, 0.239%, 0.24%, 0.241%, 0.242%, 0.243%, 0.244%, 0.245%, 0.246%, 0.247%, 0.248%, 0.249%, 0.25%, 0.251%, 0.252%, 0.253%, 0.254%, 0.255%, 0.256%, 0.257%, 0.258%, 0.259%, 0.26%, 0.261%, 0.262%, 0.263%, 0.264%, 0.265%, 0.266%, 0.267%, 0.268%, 0.269%, 0.27%, 0.271%, 0.272%, 0.273%, 0.274%, 0.275%, 0.276%, 0.277%, 0.278%, 0.279%, 0.28%, 0.281%, 0.282%, 0.283%, 0.284%, 0.285%, 0.286%, 0.287%, 0.288%, 0.289%, 0.29%, 0.291%, 0.292%, 0.293%, 0.294%, 0.295%, 0.296%, 0.297%, 0.298%, 0.299%, 0.3%, 0.301%, 0.302%, 0.303%, 0.304%, 0.305%, 0.306%, 0.307%, 0.308%, 0.309%, 0.31%, 0.311%, 0.312%, 0.313%, 0.314%, 0.315%, 0.316%, 0.317%, 0.318%, 0.319%, 0.32%, 0.321%, 0.322%, 0.323%, 0.324%, 0.325%, 0.326%, 0.327%, 0.328%, 0.329%, 0.33%, 0.331%, 0.332%, 0.333%, 0.334%, 0.335%, 0.336%, 0.337%, 0.338%, 0.339%, 0.34%, 0.341%, 0.342%, 0.343%, 0.344%, 0.345%, 0.346%, 0.347%, 0.348%, 0.349%, 0.35%, 0.351%, 0.352%, 0.353%, 0.354%, 0.355%, 0.356%, 0.357%, 0.358%, 0.359%, 0.36%, 0.361%, 0.362%, 0.363%, 0.364%, 0.365%, 0.366%, 0.367%, 0.368%, 0.369%, 0.37%, 0.371%, 0.372%, 0.373%, 0.374%, 0.375%, 0.376%, 0.377%, 0.378%, 0.379%, 0.38%, 0.381%, 0.382%, 0.383%, 0.384%, 0.385%, 0.386%, 0.387%, 0.388%, 0.389%, 0.39%, 0.391%, 0.392%, 0.393%, 0.394%, 0.395%, 0.396%, 0.397%, 0.398%, 0.399%, 0.4%, 0.401%, 0.402%, 0.403%, 0.404%, 0.405%, 0.406%, 0.407%, 0.408%, 0.409%, 0.41%, 0.411%, 0.412%, 0.413%, 0.414%, 0.415%, 0.416%, 0.417%, 0.418%, 0.419%, 0.42%, 0.421%, 0.422%, 0.423%, 0.424%, 0.425%, 0.426%, 0.427%, 0.428%, 0.429%, 0.43%, 0.431%, 0.432%, 0.433%, 0.434%, 0.435%, 0.436%, 0.437%, 0.438%, 0.439%, 0.44%, 0.441%, 0.442%, 0.443%, 0.444%, 0.445%, 0.446%, 0.447%, 0.448%, 0.449%, 0.45%, 0.451%, 0.452%, 0.453%, 0.454%, 0.455%, 0.456%, 0.457%, 0.458%, 0.459%, 0.46%, 0.461%, 0.462%, 0.463%, 0.464%, 0.465%, 0.466%, 0.467%, 0.468%, 0.469%, 0.47%, 0.471%, 0.472%, 0.473%, 0.474%, 0.475%, 0.476%, 0.477%, 0.478%, 0.479%, 0.48%, 0.481%, 0.482%, 0.483%, 0.484%, 0.485%, 0.486%, 0.487%, 0.488%, 0.489%, 0.49%, 0.491%, 0.492%, 0.493%, 0.494%, 0.495%, 0.496%, 0.497%, 0.498%, 0.499%, 0.5%, 0.501%, 0.502%, 0.503%, 0.504%, 0.505%, 0.506%, 0.507%, 0.508%, 0.509%, 0.51%, 0.511%, 0.512%, 0.513%, 0.514%, 0.515%, 0.516%, 0.517%, 0.518%, 0.519%, 0.52%, 0.521%, 0.522%, 0.523%, 0.524%, 0.525%, 0.526%, 0.527%, 0.528%, 0.529%, 0.53%, 0.531%, 0.532%, 0.533%, 0.534%, 0.535%, 0.536%, 0.537%, 0.538%, 0.539%, 0.54%, 0.541%, 0.542%, 0.543%, 0.544%, 0.545%, 0.546%, 0.547%, 0.548%, 0.549%, 0.55%, 0.551%, 0.552%, 0.553%, 0.554%, 0.555%, 0.556%, 0.557%, 0.558%, 0.559%, 0.56%, 0.561%, 0.562%, 0.563%, 0.564%, 0.565%, 0.566%, 0.567%, 0.568%, 0.569%, 0.57%, 0.571%, 0.572%, 0.573%, 0.574%, 0.575%, 0.576%, 0.577%, 0.578%, 0.579%, 0.58%, 0.581%, 0.582%, 0.583%, 0.584%, 0.585%, 0.586%, 0.587%, 0.588%, 0.589%, 0.59%, 0.591%, 0.592%, 0.593%, 0.594%, 0.595%, 0.596%, 0.597%, 0.598%, 0.599%, 0.6%, 0.601%, 0.602%, 0.603%, 0.604%, 0.605%, 0.606%, 0.607%, 0.608%, 0.609%, 0.61%, 0.611%, 0.612%, 0.613%, 0.614%, 0.615%, 0.616%, 0.617%, 0.618%, 0.619%, 0.62%, 0.621%, 0.622%, 0.623%, 0.624%, 0.625%, 0.626%, 0.627%, 0.628%, 0.629%, 0.63%, 0.631%, 0.632%, 0.633%, 0.634%, 0.635%, 0.636%, 0.637%, 0.638%, 0.639%, 0.64%, 0.641%, 0.642%, 0.643%, 0.644%, 0.645%, 0.646%, 0.647%, 0.648%, 0.649%, 0.65%, 0.651%, 0.652%, 0.653%, 0.654%, 0.655%, 0.656%, 0.657%, 0.658%, 0.659%, 0.66%, 0.661%, 0.662%, 0.663%, 0.664%, 0.665%, 0.666%, 0.667%, 0.668%, 0.669%, 0.67%, 0.671%, 0.672%, 0.673%, 0.674%, 0.675%, 0.676%, 0.677%, 0.678%, 0.679%, 0.68%, 0.681%, 0.682%, 0.683%, 0.684%, 0.685%, 0.686%, 0.687%, 0.688%, 0.689%, 0.69%, 0.691%, 0.692%, 0.693%, 0.694%, 0.695%, 0.696%, 0.697%, 0.698%, 0.699%, 0.7%, 0.701%, 0.702%, 0.703%, 0.704%, 0.705%, 0.706%, 0.707%, 0.708%, 0.709%, 0.71%, 0.711%, 0.712%, 0.713%, 0.714%, 0.715%, 0.716%, 0.717%, 0.718%, 0.719%, 0.72%, 0.721%, 0.722%, 0.723%, 0.724%, 0.725%, 0.726%, 0.727%, 0.728%, 0.729%, 0.73%, 0.731%, 0.732%, 0.733%, 0.734%, 0.735%, 0.736%, 0.737%, 0.738%, 0.739%, 0.74%, 0.741%, 0.742%, 0.743%, 0.744%, 0.745%, 0.746%, 0.747%, 0.748%, 0.749%, 0.75%, 0.751%, 0.752%, 0.753%, 0.754%, 0.755%, 0.756%, 0.757%, 0.758%, 0.759%, 0.76%, 0.761%, 0.762%, 0.763%, 0.764%, 0.765%, 0.766%, 0.767%, 0.768%, 0.769%, 0.77%, 0.771%, 0.772%, 0.773%, 0.774%, 0.775%, 0.776%, 0.777%, 0.778%, 0.779%, 0.78%, 0.781%, 0.782%, 0.783%, 0.784%, 0.785%, 0.786%, 0.787%, 0.788%, 0.789%, 0.79%, 0.791%, 0.792%, 0.793%, 0.794%, 0.795%, 0.796%, 0.797%, 0.798%, 0.799%, 0.8%, 0.801%, 0.802%, 0.803%, 0.804%, 0.805%, 0.806%, 0.807%, 0.808%, 0.809%, 0.81%, 0.811%, 0.812%, 0.813%, 0.814%, 0.815%, 0.816%, 0.817%, 0.818%, 0.819%, 0.82%, 0.821%, 0.822%, 0.823%, 0.824%, 0.825%, 0.826%, 0.827%, 0.828%, 0.829%, 0.83%, 0.831%, 0.832%, 0.833%, 0.834%, 0.835%, 0.836%, 0.837%, 0.838%, 0.839%, 0.84%, 0.841%, 0.842%, 0.843%, 0.844%, 0.845%, 0.846%, 0.847%, 0.848%, 0.849%, 0.85%, 0.851%, 0.852%, 0.853%, 0.854%, 0.855%, 0.856%, 0.857%, 0.858%, 0.859%, 0.86%, 0.861%, 0.862%, 0.863%, 0.864%, 0.865%, 0.866%, 0.867%, 0.868%, 0.869%, 0.87%, 0.871%, 0.872%, 0.873%, 0.874%, 0.875%, 0.876%, 0.877%, 0.878%, 0.879%, 0.88%, 0.881%, 0.882%, 0.883%, 0.884%, 0.885%, 0.886%, 0.887%, 0.888%, 0.889%, 0.89%, 0.891%, 0.892%, 0.893%, 0.894%, 0.895%, 0.896%, 0.897%, 0.898%, 0.899%, 0.9%, 0.901%, 0.902%, 0.903%, 0.904%, 0.905%, 0.906%, 0.907%, 0.908%, 0.909%, 0.91%, 0.911%, 0.912%, 0.913%, 0.914%, 0.915%, 0.916%, 0.917%, 0.918%, 0.919%, 0.92%, 0.921%, 0.922%, 0.923%, 0.924%, 0.925%, 0.926%, 0.927%, 0.928%, 0.929%, 0.93%, 0.931%, 0.932%, 0.933%, 0.934%, 0.935%, 0.936%, 0.937%, 0.938%, 0.939%, 0.94%, 0.941%, 0.942%, 0.943%, 0.944%, 0.945%, 0.946%, 0.947%, 0.948%, 0.949%, 0.95%, 0.951%, 0.952%, 0.953%, 0.954%, 0.955%, 0.956%, 0.957%, 0.958%, 0.959%, 0.96%, 0.961%, 0.962%, 0.963%, 0.964%, 0.965%, 0.966%, 0.967%, 0.968%, 0.969%, 0.97%, 0.971%, 0.972%, 0.973%, 0.974%, 0.975%, 0.976%, 0.977%, 0.978%, 0.979%, 0.98%, 0.981%, 0.982%, 0.983%, 0.984%, 0.985%, 0.986%, 0.987%, 0.988%, 0.989%, 0.99%, 0.991%, 0.992%, 0.993%, 0.994%, 0.995%, 0.996%, 0.997%, 0.998%, 0.999%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, or more increase in growth rate, body weight gain, feed efficiency, or any combination thereof.

In certain example embodiments, after administration of the effective amount of the stilbene composition the avian exhibits 0.001%-1% or more decrease in feed conversion ratio, such as 0.001%, to/or 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.031%, 0.032%, 0.033%, 0.034%, 0.035%, 0.036%, 0.037%, 0.038%, 0.039%, 0.04%, 0.041%, 0.042%, 0.043%, 0.044%, 0.045%, 0.046%, 0.047%, 0.048%, 0.049%, 0.05%, 0.051%, 0.052%, 0.053%, 0.054%, 0.055%, 0.056%, 0.057%, 0.058%, 0.059%, 0.06%, 0.061%, 0.062%, 0.063%, 0.064%, 0.065%, 0.066%, 0.067%, 0.068%, 0.069%, 0.07%, 0.071%, 0.072%, 0.073%, 0.074%, 0.075%, 0.076%, 0.077%, 0.078%, 0.079%, 0.08%, 0.081%, 0.082%, 0.083%, 0.084%, 0.085%, 0.086%, 0.087%, 0.088%, 0.089%, 0.09%, 0.091%, 0.092%, 0.093%, 0.094%, 0.095%, 0.096%, 0.097%, 0.098%, 0.099%, 0.1%, 0.101%, 0.102%, 0.103%, 0.104%, 0.105%, 0.106%, 0.107%, 0.108%, 0.109%, 0.11%, 0.111%, 0.112%, 0.113%, 0.114%, 0.115%, 0.116%, 0.117%, 0.118%, 0.119%, 0.12%, 0.121%, 0.122%, 0.123%, 0.124%, 0.125%, 0.126%, 0.127%, 0.128%, 0.129%, 0.13%, 0.131%, 0.132%, 0.133%, 0.134%, 0.135%, 0.136%, 0.137%, 0.138%, 0.139%, 0.14%, 0.141%, 0.142%, 0.143%, 0.144%, 0.145%, 0.146%, 0.147%, 0.148%, 0.149%, 0.15%, 0.151%, 0.152%, 0.153%, 0.154%, 0.155%, 0.156%, 0.157%, 0.158%, 0.159%, 0.16%, 0.161%, 0.162%, 0.163%, 0.164%, 0.165%, 0.166%, 0.167%, 0.168%, 0.169%, 0.17%, 0.171%, 0.172%, 0.173%, 0.174%, 0.175%, 0.176%, 0.177%, 0.178%, 0.179%, 0.18%, 0.181%, 0.182%, 0.183%, 0.184%, 0.185%, 0.186%, 0.187%, 0.188%, 0.189%, 0.19%, 0.191%, 0.192%, 0.193%, 0.194%, 0.195%, 0.196%, 0.197%, 0.198%, 0.199%, 0.2%, 0.201%, 0.202%, 0.203%, 0.204%, 0.205%, 0.206%, 0.207%, 0.208%, 0.209%, 0.21%, 0.211%, 0.212%, 0.213%, 0.214%, 0.215%, 0.216%, 0.217%, 0.218%, 0.219%, 0.22%, 0.221%, 0.222%, 0.223%, 0.224%, 0.225%, 0.226%, 0.227%, 0.228%, 0.229%, 0.23%, 0.231%, 0.232%, 0.233%, 0.234%, 0.235%, 0.236%, 0.237%, 0.238%, 0.239%, 0.24%, 0.241%, 0.242%, 0.243%, 0.244%, 0.245%, 0.246%, 0.247%, 0.248%, 0.249%, 0.25%, 0.251%, 0.252%, 0.253%, 0.254%, 0.255%, 0.256%, 0.257%, 0.258%, 0.259%, 0.26%, 0.261%, 0.262%, 0.263%, 0.264%, 0.265%, 0.266%, 0.267%, 0.268%, 0.269%, 0.27%, 0.271%, 0.272%, 0.273%, 0.274%, 0.275%, 0.276%, 0.277%, 0.278%, 0.279%, 0.28%, 0.281%, 0.282%, 0.283%, 0.284%, 0.285%, 0.286%, 0.287%, 0.288%, 0.289%, 0.29%, 0.291%, 0.292%, 0.293%, 0.294%, 0.295%, 0.296%, 0.297%, 0.298%, 0.299%, 0.3%, 0.301%, 0.302%, 0.303%, 0.304%, 0.305%, 0.306%, 0.307%, 0.308%, 0.309%, 0.31%, 0.311%, 0.312%, 0.313%, 0.314%, 0.315%, 0.316%, 0.317%, 0.318%, 0.319%, 0.32%, 0.321%, 0.322%, 0.323%, 0.324%, 0.325%, 0.326%, 0.327%, 0.328%, 0.329%, 0.33%, 0.331%, 0.332%, 0.333%, 0.334%, 0.335%, 0.336%, 0.337%, 0.338%, 0.339%, 0.34%, 0.341%, 0.342%, 0.343%, 0.344%, 0.345%, 0.346%, 0.347%, 0.348%, 0.349%, 0.35%, 0.351%, 0.352%, 0.353%, 0.354%, 0.355%, 0.356%, 0.357%, 0.358%, 0.359%, 0.36%, 0.361%, 0.362%, 0.363%, 0.364%, 0.365%, 0.366%, 0.367%, 0.368%, 0.369%, 0.37%, 0.371%, 0.372%, 0.373%, 0.374%, 0.375%, 0.376%, 0.377%, 0.378%, 0.379%, 0.38%, 0.381%, 0.382%, 0.383%, 0.384%, 0.385%, 0.386%, 0.387%, 0.388%, 0.389%, 0.39%, 0.391%, 0.392%, 0.393%, 0.394%, 0.395%, 0.396%, 0.397%, 0.398%, 0.399%, 0.4%, 0.401%, 0.402%, 0.403%, 0.404%, 0.405%, 0.406%, 0.407%, 0.408%, 0.409%, 0.41%, 0.411%, 0.412%, 0.413%, 0.414%, 0.415%, 0.416%, 0.417%, 0.418%, 0.419%, 0.42%, 0.421%, 0.422%, 0.423%, 0.424%, 0.425%, 0.426%, 0.427%, 0.428%, 0.429%, 0.43%, 0.431%, 0.432%, 0.433%, 0.434%, 0.435%, 0.436%, 0.437%, 0.438%, 0.439%, 0.44%, 0.441%, 0.442%, 0.443%, 0.444%, 0.445%, 0.446%, 0.447%, 0.448%, 0.449%, 0.45%, 0.451%, 0.452%, 0.453%, 0.454%, 0.455%, 0.456%, 0.457%, 0.458%, 0.459%, 0.46%, 0.461%, 0.462%, 0.463%, 0.464%, 0.465%, 0.466%, 0.467%, 0.468%, 0.469%, 0.47%, 0.471%, 0.472%, 0.473%, 0.474%, 0.475%, 0.476%, 0.477%, 0.478%, 0.479%, 0.48%, 0.481%, 0.482%, 0.483%, 0.484%, 0.485%, 0.486%, 0.487%, 0.488%, 0.489%, 0.49%, 0.491%, 0.492%, 0.493%, 0.494%, 0.495%, 0.496%, 0.497%, 0.498%, 0.499%, 0.5%, 0.501%, 0.502%, 0.503%, 0.504%, 0.505%, 0.506%, 0.507%, 0.508%, 0.509%, 0.51%, 0.511%, 0.512%, 0.513%, 0.514%, 0.515%, 0.516%, 0.517%, 0.518%, 0.519%, 0.52%, 0.521%, 0.522%, 0.523%, 0.524%, 0.525%, 0.526%, 0.527%, 0.528%, 0.529%, 0.53%, 0.531%, 0.532%, 0.533%, 0.534%, 0.535%, 0.536%, 0.537%, 0.538%, 0.539%, 0.54%, 0.541%, 0.542%, 0.543%, 0.544%, 0.545%, 0.546%, 0.547%, 0.548%, 0.549%, 0.55%, 0.551%, 0.552%, 0.553%, 0.554%, 0.555%, 0.556%, 0.557%, 0.558%, 0.559%, 0.56%, 0.561%, 0.562%, 0.563%, 0.564%, 0.565%, 0.566%, 0.567%, 0.568%, 0.569%, 0.57%, 0.571%, 0.572%, 0.573%, 0.574%, 0.575%, 0.576%, 0.577%, 0.578%, 0.579%, 0.58%, 0.581%, 0.582%, 0.583%, 0.584%, 0.585%, 0.586%, 0.587%, 0.588%, 0.589%, 0.59%, 0.591%, 0.592%, 0.593%, 0.594%, 0.595%, 0.596%, 0.597%, 0.598%, 0.599%, 0.6%, 0.601%, 0.602%, 0.603%, 0.604%, 0.605%, 0.606%, 0.607%, 0.608%, 0.609%, 0.61%, 0.611%, 0.612%, 0.613%, 0.614%, 0.615%, 0.616%, 0.617%, 0.618%, 0.619%, 0.62%, 0.621%, 0.622%, 0.623%, 0.624%, 0.625%, 0.626%, 0.627%, 0.628%, 0.629%, 0.63%, 0.631%, 0.632%, 0.633%, 0.634%, 0.635%, 0.636%, 0.637%, 0.638%, 0.639%, 0.64%, 0.641%, 0.642%, 0.643%, 0.644%, 0.645%, 0.646%, 0.647%, 0.648%, 0.649%, 0.65%, 0.651%, 0.652%, 0.653%, 0.654%, 0.655%, 0.656%, 0.657%, 0.658%, 0.659%, 0.66%, 0.661%, 0.662%, 0.663%, 0.664%, 0.665%, 0.666%, 0.667%, 0.668%, 0.669%, 0.67%, 0.671%, 0.672%, 0.673%, 0.674%, 0.675%, 0.676%, 0.677%, 0.678%, 0.679%, 0.68%, 0.681%, 0.682%, 0.683%, 0.684%, 0.685%, 0.686%, 0.687%, 0.688%, 0.689%, 0.69%, 0.691%, 0.692%, 0.693%, 0.694%, 0.695%, 0.696%, 0.697%, 0.698%, 0.699%, 0.7%, 0.701%, 0.702%, 0.703%, 0.704%, 0.705%, 0.706%, 0.707%, 0.708%, 0.709%, 0.71%, 0.711%, 0.712%, 0.713%, 0.714%, 0.715%, 0.716%, 0.717%, 0.718%, 0.719%, 0.72%, 0.721%, 0.722%, 0.723%, 0.724%, 0.725%, 0.726%, 0.727%, 0.728%, 0.729%, 0.73%, 0.731%, 0.732%, 0.733%, 0.734%, 0.735%, 0.736%, 0.737%, 0.738%, 0.739%, 0.74%, 0.741%, 0.742%, 0.743%, 0.744%, 0.745%, 0.746%, 0.747%, 0.748%, 0.749%, 0.75%, 0.751%, 0.752%, 0.753%, 0.754%, 0.755%, 0.756%, 0.757%, 0.758%, 0.759%, 0.76%, 0.761%, 0.762%, 0.763%, 0.764%, 0.765%, 0.766%, 0.767%, 0.768%, 0.769%, 0.77%, 0.771%, 0.772%, 0.773%, 0.774%, 0.775%, 0.776%, 0.777%, 0.778%, 0.779%, 0.78%, 0.781%, 0.782%, 0.783%, 0.784%, 0.785%, 0.786%, 0.787%, 0.788%, 0.789%, 0.79%, 0.791%, 0.792%, 0.793%, 0.794%, 0.795%, 0.796%, 0.797%, 0.798%, 0.799%, 0.8%, 0.801%, 0.802%, 0.803%, 0.804%, 0.805%, 0.806%, 0.807%, 0.808%, 0.809%, 0.81%, 0.811%, 0.812%, 0.813%, 0.814%, 0.815%, 0.816%, 0.817%, 0.818%, 0.819%, 0.82%, 0.821%, 0.822%, 0.823%, 0.824%, 0.825%, 0.826%, 0.827%, 0.828%, 0.829%, 0.83%, 0.831%, 0.832%, 0.833%, 0.834%, 0.835%, 0.836%, 0.837%, 0.838%, 0.839%, 0.84%, 0.841%, 0.842%, 0.843%, 0.844%, 0.845%, 0.846%, 0.847%, 0.848%, 0.849%, 0.85%, 0.851%, 0.852%, 0.853%, 0.854%, 0.855%, 0.856%, 0.857%, 0.858%, 0.859%, 0.86%, 0.861%, 0.862%, 0.863%, 0.864%, 0.865%, 0.866%, 0.867%, 0.868%, 0.869%, 0.87%, 0.871%, 0.872%, 0.873%, 0.874%, 0.875%, 0.876%, 0.877%, 0.878%, 0.879%, 0.88%, 0.881%, 0.882%, 0.883%, 0.884%, 0.885%, 0.886%, 0.887%, 0.888%, 0.889%, 0.89%, 0.891%, 0.892%, 0.893%, 0.894%, 0.895%, 0.896%, 0.897%, 0.898%, 0.899%, 0.9%, 0.901%, 0.902%, 0.903%, 0.904%, 0.905%, 0.906%, 0.907%, 0.908%, 0.909%, 0.91%, 0.911%, 0.912%, 0.913%, 0.914%, 0.915%, 0.916%, 0.917%, 0.918%, 0.919%, 0.92%, 0.921%, 0.922%, 0.923%, 0.924%, 0.925%, 0.926%, 0.927%, 0.928%, 0.929%, 0.93%, 0.931%, 0.932%, 0.933%, 0.934%, 0.935%, 0.936%, 0.937%, 0.938%, 0.939%, 0.94%, 0.941%, 0.942%, 0.943%, 0.944%, 0.945%, 0.946%, 0.947%, 0.948%, 0.949%, 0.95%, 0.951%, 0.952%, 0.953%, 0.954%, 0.955%, 0.956%, 0.957%, 0.958%, 0.959%, 0.96%, 0.961%, 0.962%, 0.963%, 0.964%, 0.965%, 0.966%, 0.967%, 0.968%, 0.969%, 0.97%, 0.971%, 0.972%, 0.973%, 0.974%, 0.975%, 0.976%, 0.977%, 0.978%, 0.979%, 0.98%, 0.981%, 0.982%, 0.983%, 0.984%, 0.985%, 0.986%, 0.987%, 0.988%, 0.989%, 0.99%, 0.991%, 0.992%, 0.993%, 0.994%, 0.995%, 0.996%, 0.997%, 0.998%, 0.999%, or 1% or more.

In some embodiments, the effective amount of the stilbene composition can improve the health of a gut microbiome and/or promote a healthy microbiome structure. As used herein “microbiome structure” refers to the profile of microbial species and subspecies present in the microbiome. In some embodiments, the effective amount of the stilbene composition increases the number of non-pathogenic and/or beneficial bacteria and decreases the number of pathogenic bacteria thus promoting a healthy microbiome structure. In some embodiments, the health and/or functionality of the microbes in the microbiome are improved. For example, the effective amount of the stilbene composition can improve cellular processes like DNA repair and recombination proteins and fatty acid biosynthesis, as well as improved metabolic processes like carbohydrate metabolism and energy metabolism relative to the naive system (no-injection control) (see e.g., FIG. 6A-6D).

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Polyphenols are a class of bioactive compounds of plant origin that may play an important part in reducing the risk of chronic diseases [1]. Polyphenols are naturally produced by plants as secondary metabolites and are generally involved in plant defense pathways. Recently, there has been growing scientific attention for the health benefits of dietary polyphenols. Epidemiological studies on polyphenol consumption have yielded promising evidence for polyphenols possessing health-promoting properties. These include protection against cardiovascular diseases and diabetes, likely secondary to antioxidant and anti-aging properties [2]. In a previous epidemiological study that focused on grape-originating polyphenols, researchers devised the expression “the French Paradox” to describe the observed lower coronary heart disease (CHD) mortality in French people despite their high saturated fat intake [3]. It has been postulated that moderate wine consumption provides a significant amount of dietary polyphenols, thus lowering the incidence of CHD, which may explain this dichotomy.

Resveratrol is a stilbene, a polyphenolic subclass primarily found in grapes [4]. Resveratrol has demonstrated chemoprotective, cardioprotective, anti-inflammatory and neuroprotective activities, likely due to its ability to scavenge free radicals and chelate both redox-active and redox-inactive metals [5,6]. Grapes are the most produced fruit crop in the United States. Globally, over 77.8 million tons of grapes are produced annually, with 57% being wine grapes, 36% being table grapes and 7% being dried grapes [7]. Therefore, as grape production and consumption increases, it is imperative to maximize understanding of the specific health effects of bioactive compounds that are found in this fruit. Pterostilbene is another stilbene and an analog of resveratrol present in smaller quantities within grapes and grape products, including wine [8,9]. The two methoxy substituents in pterostilbene increase the lipophilicity of the compound relative to resveratrol, which contains two hydroxy substituents. In the plant, these compounds serve as the phytoalexins found in grape skins and stems [10]. Pterostilbene is more easily absorbed than resveratrol in the gut lumen because it exhibits higher bioavailability (80%, compared with 20% from resveratrol) [11,12].

Although bioactive polyphenols can chelate iron (Fe) and zinc (Zn), studies using various in vitro and in vivo models to evaluate the effects of dietary stilbenes on mineral metabolism are not elucidated [13,14]. This is especially the case with resveratrol and pterostilbene, as there is no substantial research which has evaluated the effects of these compounds on duodenal brush border membrane (BBM) functionality or the gastro intestinal tract microbiota, which play a major role in mineral bioavailability and metabolism [15].

This Example utilizes the Gallus gallus in vivo model [16,17,18,19,20,21,22,23], to evaluate the effects of the intra-amniotic administration of stilbenes (resveratrol and pterostilbene) on the multiple criterion that influence mineral bioavailability and absorption, the mineral-dependent proteins' gene expression and BBM morphology. Additionally, cecal content was utilized for 16S rRNA gene sequencing, and potential alterations in the gut microbiota structure and function related to the administration of stilbenes were assessed. This Example evidences that intra-amniotic administration of resveratrol and pterostilbene, and the combination of both for a synergistic assessment, will lead to increased mineral absorption through favorable alterations in BBM functionality and tissue morphology and beneficially modulate the intestinal microbial populations.

### Materials and Methods

### 2.1. Sample Preparation

Resveratrol (>99%) and pterostilbene (>97%) (Sigma-Aldrich, St. Louis, MO, USA) were used for the intra-amniotic administration experiment. In addition, three grape (Vitis vinifera) variety samples (Cabernet Franc, Concord, and Noiret) were obtained from the Cornell University vineyards in Lansing and Geneva, NY. The grapes were harvested in bunches. With berries still on the stems and prior to separation, the samples were immediately frozen at −80° C. Before the analysis, the whole grapes were crushed, lyophilized and then ground by taking fruit from each bundle evenly.

### 2.2. Polyphenols Analysis

### 2.2.1. Grape Sample Preparation

Each ground whole grapes sample (0.5 g) was added to 5 mL of methanol:H₂O (50:50 v/v). The mixture was shaken for 1 min and incubated in a water bath at 24° C. for 20 min, after which the samples were vortexed, kept on Rocker (Labnet Int., Inc., Edison, NJ, USA) for 60 min, and centrifugated (4000× g for 15 min). The supernatants were removed, filtered (0.2 μm Teflon™ syringe filter) and kept at −20° C. [24,25,26].

### 2.2.2. Liquid Chromatography-Mass Spectrometry (LC-MS) Analysis

The sample extracts and polyphenol standards were diluted to 30% methanol for injection, and the compounds were separated with an ACQUITY BEH C18 column (2.1×100 mm, 1.7 μm) at 50° C. (Waters, Milford, MA, USA). Two-component and two-segment gradient elution were employed. The two components of the gradient were composed of 0.1% aqueous formic acid (FA) as Eluent A and 0.1% FA in methanol as Eluent B. The flow rate was 0.5 mL/min. The initial conditions were 5% Eluent B. The first gradient segment was linear over 3.5 min from 5% to 30% Eluent B, followed by a second linear segment from 30% to 60% Eluent B over 5.5 min. The column was rinsed at 90% Eluent B for 0.5 min and re-equilibrated at 5% Eluent B prior to the next injection. The column was fixed to a photodiode array detector (PDA) (Waters, Milford, MA, USA) to measure the UV absorbance peaks in the standards and grape samples in the range of 225-498 nm. Flow was then directed into a mass sensitive detector (QDa, Waters, Milford, MA, USA), and it was used to generate extracted ion chromatograms at the subject m/z values. The ionization parameters were set as follows: a probe temperature of 550° C. and cone voltage +/−6V. The mass range investigated was 100-250 Da. The extracts were run in positive ion modes. The compounds were identified by characteristic retention times and mass spectra and compared to the standards. LC and CMS instrumentation and result analyses acquisition were monitored via Empower™ software. Individual polyphenols were identified through comparison of the m/z and LC retention timing with the standards. The MS intensity peaks from seven replications were used for creating polyphenol standard curves for stilbenes.

### 2.3. Animals and Study Design

Cornish cross-fertile broiler eggs (n =72) were purchased from a hatchery (Moyer' s Chicks, Quakertown, PA, USA). The eggs were incubated in ideal conditions at the Cornell University Animal Science poultry farm incubator. All animal protocols were approved by the Cornell University IACUC (protocol code: 2020-0077).

### Intra-Amniotic Administration

The pure resveratrol and pterostilbene were individually diluted in 18 MΩ H₂O. The intra-amniotic administration followed the methodology previously described [17,23,24,25,26,27]. On the 17th day of embryonic development, the eggs with viable embryos (n=60) were randomly allocated into 6 groups (n=10) with a similar weight frequency distribution. For the intra-amniotic treatment, 1 mL of solution per egg was injected into the amniotic fluid (via candling) with the aid of a 21-gauge needle. After the procedure, the injection sites were sealed with cellophane tape, and the eggs were distributed into six groups: (1) no injection; (2) 18 MΩ H₂O; (3) 5% inulin; (4) 5% resveratrol; (5) 5% pterostilbene; and (6) synergistic: 4.75% resveratrol and 0.25% pterostilbene. As was included in previous studies [24,25,26,27], inulin was used as a positive control, as it belongs to a class of polysaccharides that have been shown to beneficially modulate the gut microbiome when consumed. The eggs were equally distributed in each incubator location to reduce possible allocation bias. Upon hatching (21 days), the hatchlings were euthanized by CO₂ exposure, and the blood, liver, heart, pectoral muscle, small intestine and cecum were obtained.

### 2.4. Blood Analysis and Hemoglobin Measurements

Blood was collected using micro-hematocrit heparin-coated capillary tubes (Fisher Scientific, Waltham, MA, USA). The hemoglobin (Hb) concentration was assessed spectrophotometrically using the QuantiChrom™ Hb Assay (DIHB-250, BioAssay Systems, Hayward, CA, USA) and according to specific instructions.

### 2.5. Fe and Zn Content in the Serum and Liver Samples

The serum (50 μL) and liver (0.5 g) samples were mixed with 3.0 mL of a 60:40 (v/v) solution of nitric acid (HNO₃) and perchloric acid (HClO₄) and incubated overnight. After this, the samples were heated for 2 hours at 120° C. and added to 0.25 mL of 40 μg/g yttrium (internal standard), and the block temperature was increased to 145° C. for 2 h and then 190° C. for 10 min. At the end of these procedures, the samples were cooled, diluted to a total volume of 20 mL, mixed and transferred into autosampler tubes for analysis (inductively coupled plasma atomic emission spectrometer) [26,28] (Thermo, Franklin, MA, USA).

### 2.6. Isolation of the Total RNA From the Duodenum and Liver Tissue Samples

The RNA was isolated as described previously [25,26,27,29] (RNeasy Mini Kit, Qiagen Inc., Valencia, CA, USA), and 30 mg of the duodenal tissue or liver tissue (n =8) was used to extract the total RNA. The tissues were homogenized in buffer RLT® with β-mercaptoethanol (rotor-stator homogenizer). After this, the samples were centrifuged for 3 min at 8000× g, and an aliquot of the supernatant was added with 1 volume of 70% ethanol. Then, 700 μL of this solution was added to a mini column and centrifuged for 15 s at 8000x g. In new 2-mL collection tubes, the RNeasy columns of 0.5 mL of buffer RPE® were added, centrifugated for 15 s at 8000× g, and the buffer RPE®was added once more, followed by centrifugation for 2 min at 8000× g. Finally, the RNA was eluted in 50 μL of RNase-free water and quantified by absorbance at 260 and 280 nm.

The integrity of the 18S ribosomal RNAs was tested (1.5% agarose gel electrophoresis followed by ethidium bromide staining). DNA contamination was addressed by using a TURBO DNase removal kit (AMBION, Austin, TX, USA). The procedures were conducted in RNase-free conditions.

### 2. 7. Real-Time Polymerase Chain Reaction (RT-PCR) and Primer Design

RT-PCR was conducted as described [24,25,26]. The cDNA was created from a 20-μL reverse transcriptase (RT) reaction (BioRad C1000 Touch Thermocycler) and using the Improm-II Reverse Transcriptase Kit (Promega, Madison, WI, USA). The cDNA content was assessed by absorbance at 260 and 280 nm, with an extinction coefficient of 33 (single-stranded DNA). Genomic DNA contamination was evaluated via a real-time RT-PCR assay for the reference gene samples [17,18,30].

The primers used in the real-time PCR were formulated in accordance with relevant gene sequences (GenBank database), via Real-Time Primer Design Tool software (IDT DNA, Coralville, IA, USA) and as was previously described [24,25,26]. The primer sequences related to iron, zinc, calcium and magnesium metabolism, immune response, hypertension and BBM functionality that were utilized are indicated in Table 1. The specificity of the primers was verified via BLAST search versus the genomic National Center for Biotechnology Information (NCBI) database. The reference gene used was the 18S rRNA specific for the Gallus gallus model.

TABLE 1
Sequences of Experimental Primers

| | | | | | | |
|---|---|---|---|---|---|---|
| | | SEQ | | SEQ | Base | |
| | Forward | ID | Reverse | ID | Pairs | GI |
| Analyte | Primer (5′-3′) | NO: | Primer (5′-3′) | NO: | Length | Identifier |

Calcium Metabolism

| | | | | |
|---|---|---|---|---|
| TRPV6 | GCTCCCAGAACCTT | CCAGGTAATCCTGAGC | 123 | 418307 |
| | CTCTATTT | TCTAATG | | |
| PMCA1b | TGCAGATGCTGTGG | CCATAAGGCTTCCGCA | 100 | 374244 |
| | GTAAAT | ATAGA | | |
| NXC1 | CCTGACGGAGAAAT | CCCAGGAGAAGACACA | 114 | 395760 |
| | AAGGAAGA | GATAAA | | |

Iron Metabolism

| | | | | |
|---|---|---|---|---|
| DMT1 | TTGATTCAGAGCCT | GCGAGGAGTAGGCTTG | 101 | 206597489 |
| | CCCATTAG | TATTT | | |
| Ferroportin | CTCAGCAATCACTG | ACTGGGCAACTCCAGA | 98 | 61098365 |
| | GCATCA | AATAAG | | |
| DcytB | CATGTGCATTCTCT | CTCCTTGGTGACCGCA | 103 | 20380692 |
| | TCCAAAGTC | TTAT | | |

Immune Response

| | | | | |
|---|---|---|---|---|
| IL-1β | CTCACAGTCCTTCG | TGTTGAGCCTCACTTT | 119 | 88702685 |
| | ACATCTTC | CTGG | | |
| IL-6 | ACCTCATCCTCCGA | GCACTGAAACTCCTGG | 105 | 302315692 |
| | GACTTTA | TCTT | | |
| TNF-α | GACAGCCTATGCCA | TTACAGGAAGGGCAAC | 109 | 53854909 |
| | ACAAGTA | TCATC | | |

Magnesium Metabolism

| | | | | |
|---|---|---|---|---|
| MRS2 | GCTGGTAACCGGGA | GCAGGAACATGAGGAG | 105 | 420820 |
| | TTATGT | GTAAT | | |
| TRPM6 | ACAGATGCTGCTGA | AAGATAGTGGGTGGTA | 99 | 100859603 |
| | CTGATATG | GGAGAA | | |
| TRPM7 | GCGTGGGATAGAGT | TCACAAGGGCATCCAA | 100 | 427502 |
| | TGACATT | CATAG | | |

Zinc Metabolism

| | | | | |
|---|---|---|---|---|
| ZnT1 | GGTAACAGAGCTGC | GGTAACAGAGCTGCCT | 105 | 54109718 |
| | CTTAACT | TAACT | | |
| ZnT7 | GGAAGATGTCAGGA | CGAAGGACAAATTGAG | 87 | 56555152 |
| | TGGTTCA | GCAAAG | | |
| ZIP4 | TCTCCTTAGCAGAC | GTGACAAACAAGTAGG | 95 | 107050877 |
| | AATTGAG | CGAAAC | | |
| ZIP9 | CTAAGCAAGAGCAG | CATGAACTGTGGCAAC | 100 | 237874618 |
| | CAAAGAAG | GTAAAG | | |

Hypertension

| | | | | |
|---|---|---|---|---|
| ACE | CATGGCCTTGTCTG | GAGGTATCCAAAGGGC | 142 | 424059 |
| | TCTCC | AGG | | |
| ATIR | TCATCTGGCTCCTT | AACCTAGCCCAACCCT | 138 | 396065 |
| | GCTGG | CAG | | |

BBM Functionality

| | | | | |
|---|---|---|---|---|
| AP | CGTCAGCCAGTTTG | CTCTCAAAGAAGCTGA | 138 | 45382360 |
| | ACTATGTA | GGATGG | | |
| SI | CCAGCAATGCCAGC | CGGTTTCTCCTTACCA | 95 | 2246388 |
| | ATATTG | CTTCTT | | |
| SGLT1 | GCATCCTTACTCTG | TATCCGCACATCACAC | 106 | 8346783 |
| | TGGTACTG | ATCC | | |
| 18s rRNA | GCAAGACGAACTAA | TCGGAACTACGACGGT | 100 | 7262899 |
| | AGCGAAAG | ATCT | | |

TRPV6: transient receptor potential cation channel subfamily V member 6;
PMCA1b: plasma membrane Ca2+ATPase isoform 1b;
NCX1: sodium-calcium exchanger 1;
DMT1: divalent metal transporter 1;
Dcytb: duodenal cytochrome b;
IL1β: interleukin 1 beta;
IL6: interleukin 6;
TNF-α: tumor necrosis factor alpha;
MRS2: mitochondrial magnesium channel type 2;
TRPM6 and TRPM7: transient receptor potential melastatin;
ZnT and ZIP: zinc transporter proteins;
ACE: angiotensin-converting enzyme;
ATIR: angiotensin II receptor type I;
AP: amino peptidase;
SI: sucrose isomaltase;
SGLT1: sodium-glucose transport protein 1;
18S IRNA: 18S ribosomal subunit.
*Heart analysis.

2.8. Real-Time qPCR Design

The procedures were performed as previously published [24,25,26,28]. The prepared cDNA was applied in a 10-μL reaction containing 2× BioRad SSO SYBR Green Supermix (Hercules, CA, USA). Table 1 shows the primers used in this study. The optimal MgCl₂ concentration achieved, for each reaction, an amplification plot (lowest cycle product, Cp), the uppermost fluorescence intensity and the steepest amplification slope. No template control of nuclease-free water was used in order to eliminate DNA contamination. For each reaction (duplicates), 8 μL of the master mix and 2 μL cDNA were pipetted into a 96-well plate, and for the standard curve, seven points were evaluated in duplicate. The Bio-Rad CFX96 Touch (Hercules, CA, USA) system was used to amplify the double-stranded DNA under optimal PCR conditions: denaturing at 95° C. for 30 s, 40 cycles of denaturing at 95° C. for 15 s, changing annealing temperatures as indicated by Integrated DNA Technologies for 30 s and elongating at 60° C. for 30 s.

According to Bio-Rad CFX Maestro 1.1 (Version 4.1.2433.1219, Hercules, CA, USA), the Cp values based on the “second derivative maximum” were used to obtain data on the expression levels of the genes. The assays were calculated by including a standard curve in the real-time qPCR analysis, and a standard curve with four points was prepared by a 1:10 dilution (duplicatea). A graph of the Cp vs. log 10 concentrations was produced, and the efficiencies were calculated as 10 (1/slope). The specificities of the amplified real-time RT-PCR products were confirmed by melting curve analysis (60-95° C.) after 40 cycles, resulting in several different specific products with specific melting temperatures.

2.9. 16S rRNA Gene Amplification and Sequencing

As was formerly described [22], cecal samples were used to extract microbial genomic DNA (PowerSoil DNA isolation kit, MoBio Laboratories Ltd., Carlsbad, CA, USA). The V4 hypervariable region of the bacterial 16S rRNA gene was PCR-amplified per sample (515F-806R primers) and with 12-base barcodes [31]. Each PCR reaction was composed of 25 μL Primestar max PCR mix (Takara Kusatsu, Shiga, Japan), 2μM of each primer, 17 μL of ultra-pure water and a 4-μL DNA template, as indicated: a denaturing step for 3 min at 95° C., 30 cycles of 10 s at 98° C., 5 s at 55° C., 20 s at 72° C. and final elongation at 72° C. for 1 min. The PCR products were purified (Beckman Coulter, Atlanta, GA, USA) and quantified (Quant-iT PicoGreen dsDNA quantitation kit, Invitrogen, Carlsbad, CA, USA). Equimolar ratios of the samples were pooled and sequenced (Illumina, Inc., Madison, WI, USA).

16S rRNA Gene Sequence Analysis

The 16S rRNA gene sequence analysis was performed using QIIME2 as previously described [28]. The sequence reads were demultiplexed by per-sample barcodes, and the Divisive Amplicon Denoising Algorithm (DADA2) was used to correct the Illumina-sequenced amplicon read errors. Sequences from a phylogenetic tree were taxonomically classified using the Greengenes [34] reference database at a confidence threshold of 99%. The Greengenes taxonomies were used to generate summaries of the taxonomic distributions of the features across different levels (phylum, order, family and genus). Low-abundance amplicon sequence variants (ASVs) (observed only in one sample per group) were filtered out from the ASV table. The alpha and β diversity were calculated from samples containing at least 9.800 sequences. The alpha diversity parameter, a measure of the richness of the bacterial community, was calculated using the Shannon Index, and the beta diversity was assessed by a Jaccard similarity distance matrix. Linear discriminant analysis effect size (LEfSe) [35] was conducted according to the relative abundances of each bacterial community to verify the highlights that significantly differed between the samples. Metagenome functional predictive analysis of the communities was performed via

phylogenetic investigation in PICRUSt [36] software (version 1.1.3). The identified sequences' abundances were normalized by the 16S rRNA gene copy number and compared to a known phylogenetic reference tree (Greengenes database). The enriched metabolic pathways were assessed via the Kyoto Encyclopedia of Genes and Genomes (KEGG). Data indicating significant differences in the functional pathways between the groups was plotted.

### 2.10. Morphometric Examination of Duodenal Tissue

Villus epithelium analysis was conducted as previously published [29,37]. The duodenal samples were soaked in buffered formaldehyde (4% (v/v)), dehydrated, cleared and embedded in paraffin. Numerous sections were cut with a thickness of 5 μm and put on slides. The sections were deparaffinized in xylene, after which they were rehydrated in a series of graded alcohol. Finally, the slides were stained with Alcian Blue-periodic acid-Schiff and investigated under a light microscopy. The variables were assessed (light microscope, EPIX XCAP software, standard version, Olympus, Waltham, MA, USA) for the following: villus length, villus diameter, depth of crypts, goblet cell diameter, crypt goblet cell number and villus goblet cells type number (acidic, neutral or mixed). Four segments for each biological sample and five biological samples per treatment group were examined. The goblet cells were enumerated at 10 villi/sample, and the means were calculated for statistical analysis.

### 2.11. Statistical Analysis

The values in this paper were expressed as means and standard errors of the means (SEM). The experimental groups for the intra-amniotic assay were in a completely randomized order. The results were analyzed by Analysis of Variance (ANOVA). Following this, to obtain the “p-value”, a post hoc Duncan test was utilized to compare all groups. SPSS software version 26.0 was used to carry out the statistical analysis, where p<0.05 was established as the level of significance. For the microbiome results, the Shannon Index alpha diversity metric was utilized to determine the bacterial richness in the samples, and the differences among the groups were analyzed by ANOVA. For assessing β diversity, the Jaccard distances were calculated by the pairwise PERMANOVA test. Statistically significant p-values linked to microbial functions were determined by LEfSe and corrected for multiple comparisons according to the Benjamini—Hochberg false discovery rate (FDR). SAS version 9.3 (SAS Institute, Cary, NC, USA) was used for the statistics.

### Results

### 3.1. Stilbenes Compounds Identified in Grape Samples

The resveratrol, the compound used in the in ovo experiment, was shown to be found in commonly grown Vitis spp. grapes from the central New York region. However, the pterostilbene was not detected in the grape samples evaluated, and the retention time is presented in Table 2.

TABLE 2
Polyphenols present within 3 grape varieties.

| Polyphenolic | Mean | M + H | Retention Time | Concentration |
|---|---|---|---|---|
| Compounds | (Da) | (Da) | (min) | (ng/g) |
| trans- | 228.24 | 229.24 | 5.334 ± 0.008 | CF: 64.7 ± 3.9 |
| Resveratrol | | | | Noire: 60.2 ± 2.8 |
| | | | | Concord: 1.85 ± 0.3 |
| trans- | 256.3 | 257.3 | 10.341 ± 0.007 | ND |
| Pterostilbene | | | | |

### 3.2. In Ovo Assay

3.2.1. Blood hemoglobin (Hb), Serum and Hepatic Fe and Zn Concentrations

The Hb concentration for the synergistic treatment group was significantly lower (p<0.05) compared with the controls and other treatment groups. Furthermore, there was no difference between the 5% resveratrol and 5% pterostilbene groups compared with the control groups (Table 3).

TABLE 3
Blood hemoglobin (Hb) concentration and Fe and Zn contents.

| Treatment Group | Hb (g/dL) |
|---|---|
| No injection | 8.75 ± 0.80^{a} |
| 18 MΩ H₂O | 10.60 ± 0.59^{a} |
| 5% Inulin | 8.86 ± 0.69^{a} |
| 5% Resveratrol | 9.71 ± 0.53^{a} |
| 5% Pterostilbene | 8.95 ± 0.77^{a} |
| Synergistic | 8.04 ± 0.94^{b} |

The synergistic group presented higher Fe and Zn levels in the liver and in the serum compared with the no injection group (p<0.05). The 5% pterostilbene group had a significantly greater serum Zn concentration compared with the control groups and 5% resveratrol group (p<0.05) and presented no difference compared with the synergistic group (p>0.05).

### 3.2.2. Gene Expression of BBM Proteins, BBM Functionality, the Immune System and Hypertension

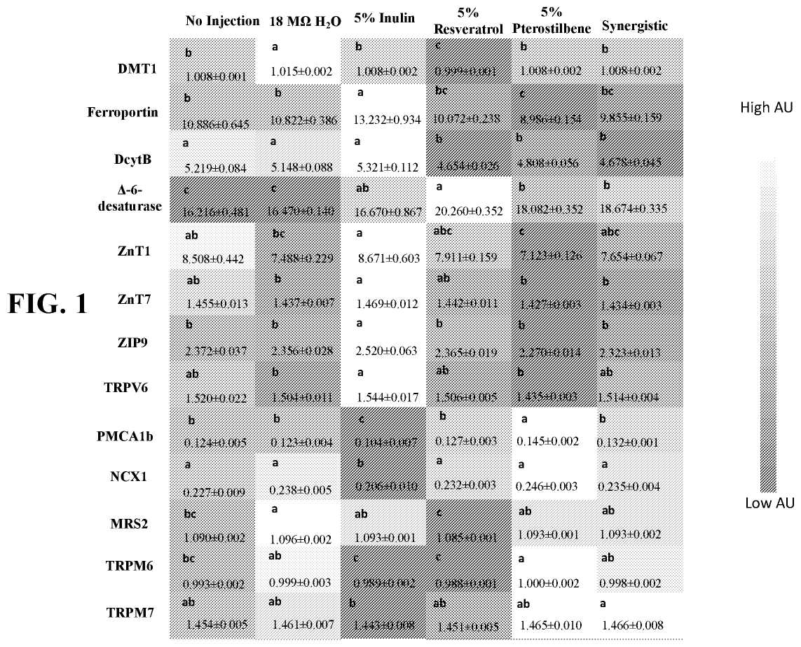

FIG. 1 shows the gene expression of various proteins indirectly involved in mineral metabolism. The expression of DcytB was significantly downregulated in all treatment groups compared with the control groups (p<0.05), and the relative expression of ferroportin was downregulated in the treatment groups compared with the 5% inulin control (p<0.05). DMT1 relative expression was the lowest in the 5% resveratrol group compared with the other treatments and the controls (p<0.05). The α-6-desaturase is involved with the fatty acid biosynthesis and is an indicator of Zn deficiency [38,39,40]. Its activity was significantly upregulated in all treatment groups compared with the no injection and 18 MΩ H₂O control groups (p<0.05), and in the 5% pterostilbene and synergistic groups, the α-6-desaturase gene expression was similar to the 5% inulin control (p>0.05).

The relative expressions of the Zn transporters and importers (ZnT1, ZnT7 and ZIPS) presented no difference (p>0.05) compared with the no injection and 18 MΩ H₂O control groups. Related to mineral absorption and the functionality of BBM proteins, after the administration of grape stilbenes, we observed an upregulation in proteins related to calcium (PMCA1b and NCX1) and magnesium metabolism (TRPM6) in the 5% pterostilbene and synergistic groups compared with the 5% inulin control group (p<0.05) but with no difference related to the other control groups (p>0.05). In addition, we did not observe differences in the relative expressions of TRPV6, MRS2 or TRPM7 in the treatment groups compared with the no injection and 18 MΩ H₂O controls (p>0.05) (FIG. 1).

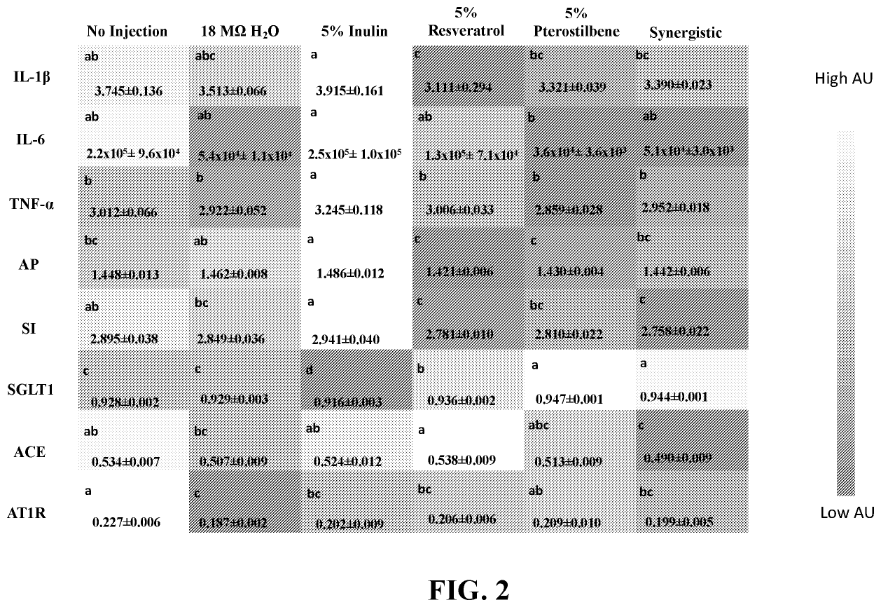

The effect of stilbenes administration on BBM proteins involved with inflammation, BBM functionality and hypertension were also evaluated (FIG. 2). The inflammatory cytokines IL-1β and TNF-α were downregulated (p<0.05) in all experimental groups (5% resveratrol, 5% pterostilbene and synergistic) compared with the 5% inulin control group. IL-6 was downregulated <0.05) in the 5% pterostilbene group compared with the 5% inulin control, but there was no difference between the other control groups (no injection or 18 MΩ H₂O) (p>0.05). Related to BBM functionality, the relative expression of AP was downregulated in the 5% resveratrol and 5% pterostilbene groups relative to the 18 MΩ M₂O and 5% inulin control groups (p<0.05), and SI was down regulated in the 5% resveratrol and synergistic groups compared with the no injection and 5% inulin control groups (p<0.05). However, it was interesting that the relative expression of SGLT1 was upregulated in all three experimental groups compared with the controls (p<0.05) (FIG. 2).

The gene expression of proteins related to the hypertension process was also evaluated, and the relative expression of ACE was downregulated in the synergistic group compared with the no injection and 5% inulin control groups (p<0.05), and AT1R gene expression was downregulated in the 5% resveratrol and synergistic groups compared with the no injection control (p<0.05) (FIG. 2).

### 3.2.3. Body Weight, Cecum Weight and Cecum: Body Weight Ratio

There were no significant differences (p<0.05) in the cecum-to-body-weight ratios between treatment groups (Table 4); the average body weight for the synergistic treatment group was significantly lower (p<0.05) than the positive controls.

TABLE 4

| Treatment | Body Weight | Cecum Weight | Cecum:Body |
|---|---|---|---|
| Group | Average (g) | Average (g) | Weight Ratio |
| No injection | 45.4 ± 1.3^{b} | 0.5 ± 0.1^{a} | 0.010 ± 0.002^{a} |
| 18 MΩ H₂O | 46.9 ± 1.2^{a} | 0.5 ± 0.1^{a} | 0.011 ± 0.001^{a} |
| 5% Inulin | 49.7 ± 0.9^{a} | 0.6 ± 0.1^{a} | 0.012 ± 0.001^{a} |
| 5% Resveratrol | 47.1 ± 1.4^{a} | 0.6 ± 0.0^{a} | 0.013 ± 0.001^{a} |
| 5% Pterostilbene | 46.7 ± 0.7^{a} | 0.6 ± 0.1^{a} | 0.013 ± 0.001^{a} |
| Synergistic | 44.8 ± 1.0^{b} | 0.6 ± 0.0^{a} | 0.013 ± 0.001^{a} |

Body weight, Cecum weight, and Cecum:Body weight ratio in all groups. Values are means ± SEM, n = 8.
^{a, b}Treatment groups not indicated by the same letter are significantly different (p < 0.05)

### 3.2.4. Iron and Zinc Concentrations in Liver and Serum

As shown in Table 5, there were significant differences (p<0.05) in the liver iron concentrations between the no-injection control and all treatment groups. Furthermore, the 5% Resveratrol and Synergistic treatment groups had similar iron concentrations to the inulin positive control, representing improved iron status. The synergistic treatment group had significant differences (p<0.05) in the liver zinc concentrations, which is a novel finding, as zinc status has not been significantly affected in previous in ovo studies [23, 26, 77]. Serum zinc concentrations were significantly greater (p<0.05) in all treatment groups than the no-injection and 18 MΩ water control groups. Serum iron concentrations were significantly greater (p<0.05) than all controls in the synergistic treatment group.

TABLE 5
Iron and zinc concentrations in liver and serum (μg/g).

| Treatment | Liver | | Serum | |
|---|---|---|---|---|
| Group | Iron (μg/g) | Zinc (μg/g) | Iron (μg/g) | Zinc (μg/g) |
| No injection | 32.47 ± 2.83 ^{b} | 15.79 ± 0.95 ^{b} | 2.09 ± 0.24 ^{b} | 0.86 ± 0.08 ^{c} |
| 18 MΩ H₂O | 37.93 ± 4.93 ^{ab} | 16.12 ± 0.96 ^{b} | 2.00 ± 0.27 ^{b} | 0.84 ± 0.07 ^{c} |
| 5% Inulin | 48.96 ± 4.39 ^{a} | 18.23 ± 0.88 ^{ab} | 2.76 ± 0.33 ^{b} | 0.97 ± 0.08 ^{bc} |
| 5% Resveratrol | 46.45 ± 4.71 ^{a} | 16.72 ± 1.58 ^{b} | 2.42 ± 0.23 ^{b} | 0.97 ± 0.12 ^{bc} |
| 5% Pterostilbene | 37.43 ± 1.52 ^{ab} | 14.43 ± 0.49 ^{b} | 2.94 ± 0.44 ^{b} | 1.33 ± 0.20 ^{a} |
| Synergistic | 50.96 ± 6.08 ^{a} | 21.22 ± 3.36 ^{a} | 3.87 ± 0.41 ^{a} | 1.24 ± 0.11 ^{ab} |

Values are means ± SEM, n = 8.
^{a, b, c} Treatment groups not indicated by the same letter are significantly different (p < 0.05)

### 3.2.5. Glycogen Analysis

There were no significant differences (p<0.05) in pectoral muscle glycogen concentration between groups (Table 6).

TABLE 6
Concentrations of glycogen in pectoral muscle.

| Treatment Group | Glycogen (mg/g) |
|---|---|
| No injection | 0.034 ± 0.011^{a} |
| 18 MΩ H₂O | 0.022 ± 0.003^{a} |
| 5% Inulin | 0.029 ± 0.005^{a} |
| 5% Resveratrol | 0.019 ± 0.004^{a} |
| 5% Pterostilbene | 0.033 ± 0.010^{a} |
| Synergistic | 0.013 ± 0.006^{a} |

Values are means ± SEM, n = 8.
^{a}, b Treatment groups not indicated by the same letter are significantly different (p < 0.05)

### 3.2.6. Liver Ferritin

There was a significantly lower (p<0.05) amount of liver ferritin in the treatment groups relative to the negative controls.

TABLE 8
Liver Ferritin.

| Treatment Group | Liver Ferritin (A.U.) |
|---|---|
| No Injection | 1.974 ± 0.005^{a} |
| 18 MΩ H₂O | 1.534 ± 0.519^{ab} |
| 5% Inulin | 0.148 ± 0.005^{c} |
| 5% Resveratrol | 0.839 ± 0.393^{bc} |
| 5% Pterostilbene | 1.011 ± 0.351^{bc} |
| Synergistic | 0.105 ± 0.013^{c} |

Values are means ± SEM, n = 5.
^{a-c}Treatment groups not indicated by the same letter are significantly different (p < 0.05)

3.2.7. Gene expression of Fe-, Zn-, Ca-, and Mg-related genes

The relative expressions of Ferroportin, and DcytB were significantly (p<0.05) downregulated in all treatment groups. DMT1 was additionally downregulated significantly in the resveratrol treatment group. The relative expression of Δ-6-desaturase was significantly upregulated (p<0.05) in all treatment groups. The relative expressions of the Zinc Transporters and Importers—ZnT1, ZnT7, and ZIPS—were all significantly downregulated relative to the positive control (p<0.05). Significant downregulation (p<0.05) of TRPV6 was observed in the pterostilbene treatment group relative to the negative controls. The relative expressions of PMCA1b and NCX1 were significantly upregulated (p<0.05) in all treatment groups relative to the controls. No significant differences (p >0.05) were observed between treatment groups and controls in the relative expressions of MRS2, TRPM6, and TRPM7.

### 3.2.8. Gene Expression of Immune and BBM Functional Genes

ACE and AT1R were significantly downregulated (p<0.05) in the synergistic treatment group indicating an anti-hypertensive effect by the treatment group. All treatment groups showed a significant inhibitory effect (p<0.05) on the inflammation marker genes: IL1β, IL6, and TNF-α. The relative expressions of AP and SI decreased significantly (p<0.05) in all stilbene treatment groups. However, the gene expression of SGLT1 increased significantly (p<0.05) in all treatment groups relative to the controls.

### 3.2.9. Duodenal Morphometric Parameters

The 5% pterostilbene and synergistic treatment groups presented elevated (p<0.05) villus lengths and diameters versus the controls and higher (p<0.05) depths of the crypts relative to the 18 MΩ H₂O and 5% inulin controls (Table 9).

TABLE 9
Effect of the intra-amniotic administration of stilbenes on the duodenal
small intestinal villi length, villi diameter and crypt depth.

| Treatment | Villus Length | Villus Diameter | Depth of Crypts | Mucus Layer Width |
|---|---|---|---|---|
| Group | (μm) | (μm) | (μm) | (μm) |
| No Injection | 235.86 ± 4.08^{e} | 52.14 ± 0.67^{b} | 66.30 ± 1.33^{a} | 11.84 ± 0.24^{a} |
| 18 MΩ H₂O | 223.29 ± 4.53^{e} | 52.52 ± 5.21^{c} | 53.42 ± 1.11^{b} | 6.43 ± 0.15^{b} |
| 5% Inulin | 262.54 ± 3.76^{d} | 44.57 ± 0.79^{d} | 51.30 ± 1.07^{b} | 5.27 ± 0.13^{c} |
| 5% Resveratrol | 294.92 ± 3.48^{c} | 54.47 ± 0.70^{b} | 63.73 ± 1.20^{a} | 4.80 ± 0.13^{d} |
| 5% Pterostilbene | 451.58 ± 11.21^{b} | 81.33 ± 4.95^{a} | 66.41 ± 1.19^{a} | 3.76 ± 0.10^{e} |
| Synergistic | 573.92 ± 9.14^{a} | 77.37 ± 0.99^{a} | 63.98 ± 1.13^{a} | 4.60 ± 0.13^{d} |

Values are the means ± SEM, n = 5.
^{a-e}Treatment groups not indicated by the same letter are significantly different (p < 0.05)

All the treatment groups demonstrated a higher (p<0.05) goblet cell diameter compared with the controls, and the synergistic group presented the highest (p<0.05) goblet cell diameter relative to all the experimental groups. However, the synergistic treatment group demonstrated a significantly lower (p<0.05) crypt goblet cell number relative to the other treatment groups and the 18 MΩ H₂O and 5% inulin controls. Furthermore, there was a significantly lower (p<0.05) amount of acidic villi goblet cells in the synergistic group and a significantly higher (p<0.05) amount of mixed villi goblet cells in the synergistic and 5% pterostilbene treatment groups relative to the controls (Table 10).

TABLE 10
Effect of the intra-amniotic administration of stilbenes on the goblet cells of the small intestine

| | Goblet Cell | | | | |
|---|---|---|---|---|---|
| Treatment | Diameter | Crypt Goblet | Villus Goblet Cell Type Number | | |
| Group | (μM) | Cell Number | Acidic | Neutral | Mixed |
| No Injection | 7.27 ± 0.05 ^{c} | 9.53 ± 0.32 ^{cd} | 10.22 ± 0.26 ^{b} | 0.12 ± 0.03 ^{a} | 0.13 ± 0.03 ^{cd} |
| 18 MΩ H₂O | 6.75 ± +0.05 ^{e} | 10.01 ± 0.22 ^{c} | 9.46 ± 0.23 ^{c} | 0.00 ± 0.00 ^{b} | 0.05 ± 0.03 ^{de} |
| 5% Inulin | 7.12 ± 0.06 ^{d} | 12.27 ± 0.33 ^{a} | 10.65 ± 0.25 ^{ab} | 0.00 ± 0.00 ^{b} | 0.02 ± 0.01 ^{e} |
| 5% Resveratrol | 7.94 ± 0.06 ^{b} | 10.91 ± 0.21 ^{b} | 10.97 ± 0.23 ^{a} | 0.00 ± 0.00 ^{b} | 0.17 ± 0.03 ^{c} |
| 5% Pterostilbene | 7.81 ± 0.07 ^{b} | 10.04 ± 0.18 ^{c} | 10.78 ± 0.22 ^{ab} | 0.00 ± 0.00 ^{b} | 0.28 ± 0.05 ^{b} |
| Synergistic | 8.55 ± 0.07 ^{a} | 9.02 ± 0.15 ^{d} | 8.29 ± 0.17 ^{d} | 0.00 ± 0.00 ^{b} | 0.66 ± 0.05 ^{a} |

Values are the means ± SEM, n = 5.
^{a-e} Treatment groups not indicated by the same letter are significantly different (p < 0.05)

### 3.3. Analysis of the Gut Microbiota

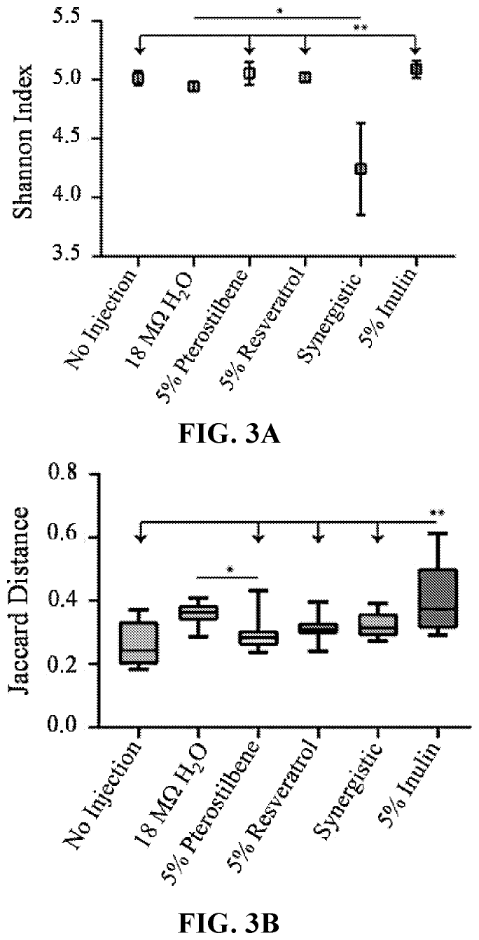

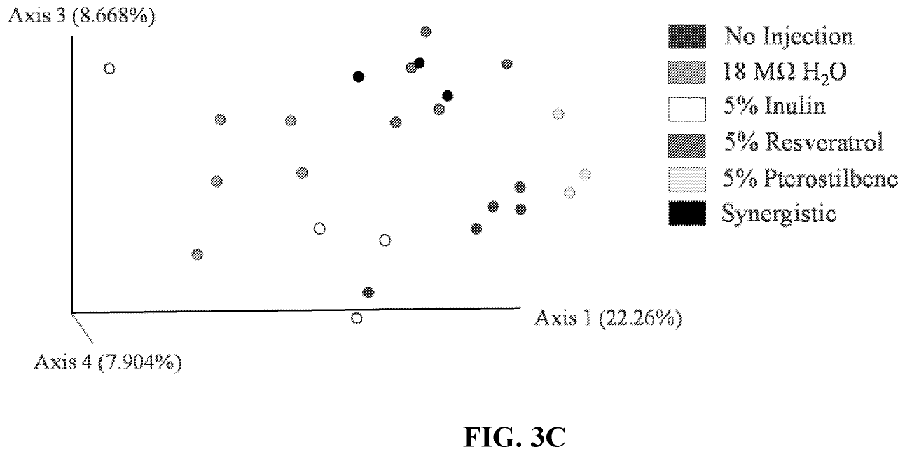

The gut microbial diversity differences among the treatment and control groups are shown in FIGS. 3A-3C. Significantly lower a diversity was identified in the synergistic group relative to the other treatment groups (p<0.05) (FIG. 3A). The β diversity indicates the variation in bacterial communities between samples (FIGS. 3B-3C). Spatial ordination of the data based their collections of sequences showed statistical differences in the distance metrics among the 5% inulin control compared with all other experimental groups (p<0.01) and a lower β diversity in the 5% pterostilbene compared with the 18 MΩ H₂O control (p<0.05). Furthermore, the β diversities of the three treatment groups with stilbenes were not significantly different (p>0.05).

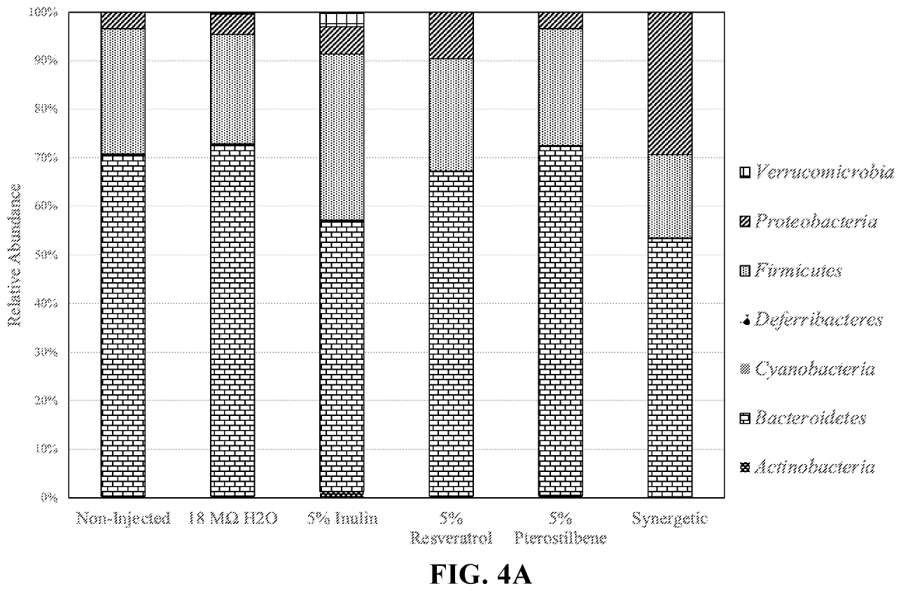

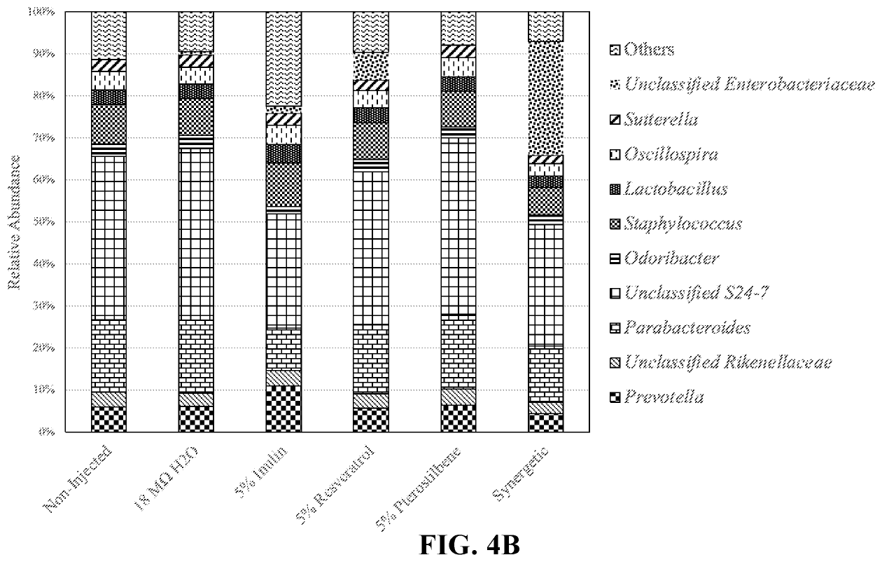

The predominant phyla in all treatment groups were Bacteroidetes and Firmicutes (FIG. 4A). In general, the cecal gut microbiome of Gallus gallus at the phylum level is very similar to that observed in the human gut, as has been previously demonstrated in human clinical trials [41]. Proteobacteria, Verrucomicrobia, Actinobacteria, Deferribacteres, and Cyanobacteria were detected in the guts of all the treatment groups in lower abundances, and there was a lower (p<0.05) abundance of Actinobacteria in the synergistic treatment group relative to the 5% inulin control. In addition, there was a tendency to reduce (p=0.083) the abundance of Firmicutes in the synergistic group relative to the 5% inulin control (FIG. 4A). At the genus level, the predominant bacteria in all treatment groups was Unclassified S24-7, belonging to a family of bacteria within the Bacteroidetes phyla. However, we did not observe a difference (p>0.05) in the bacteria composition at the genus level (FIG. 4B).

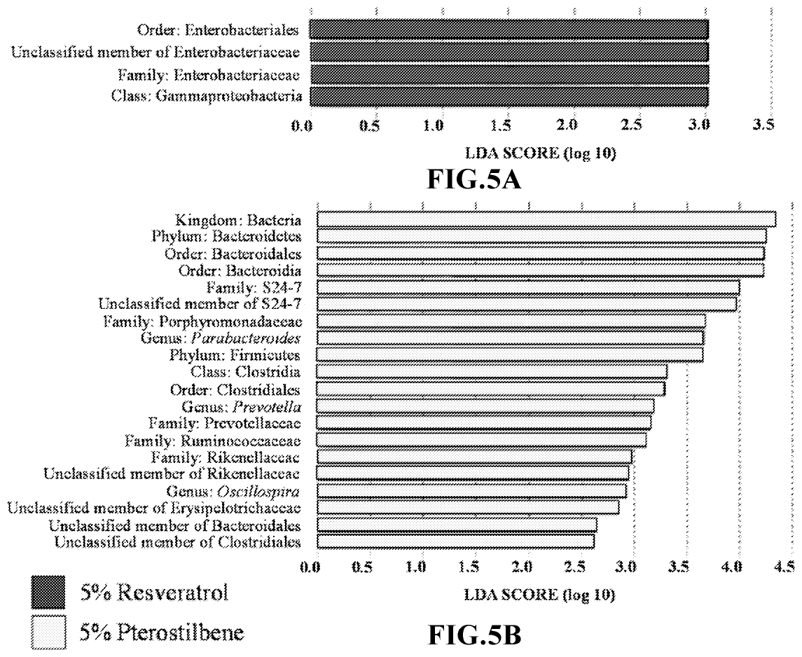

Furthermore, the linear discriminant analysis effect size method (LEfSe) was used to investigate the bacterial biomarkers and isolate gut microbiome differences between the treatment groups. The Enterobacteriales order and Enterobacteriaceae family, a member of the Proteobacteria phylum, was significantly enriched (p<0.05) in the 5% resveratrol treatment group compared with the no injection control group (FIG. 5A). However, in the 5% pterostilbene treatment group, we observed that the most differentially enriched taxa were related to members of the Bacteroidetes phylum, such as the S24-7 family, members of the Clostridiales order and the Oscillospira genus compared with the no injection control group (FIG. 5B).

To investigate any alterations in the metagenomic potential of the gut microbiota, metagenome functional predictive analysis was carried out using PICRUSt software. The feature abundance was normalized by the 16S rRNA gene copy number and identified using the Greengenes database, and the KEGG orthologs prediction was calculated. The 5% pterostilbene group demonstrated significant enrichment (p<0.05) of all the KEGG metabolic pathways identified compared with the no injection, 18 MΩ H₂O and 5% inulin controls, except by metabolic processes related to the bacterial secretion system, ion channels and carbohydrate and fatty acid metabolism. However, there was no difference (p>0.05) in the KEGG metabolic pathways between the 5% resveratrol and synergistic treatment groups compared with the controls (FIGS. 6A-6D).

### 4. Discussion

Stilbenes, such as resveratrol and pterostilbene, are plant-bioactive compounds primarily found in grapes. These compounds present potent proposed health benefits and may play an essential role in reducing the risk of chronic diseases, protection against cardiovascular diseases and diabetes [42,43,44]. In the plant, stilbenes serve as phytoalexins, which are defensive substances produced in response to infections [45]. Previous studies, which evaluated the benefits of resveratrol and pterostilbene, have posited chemoprotective [46], cardioprotective and anti-inflammatory activities [42,43,44,47]. However, limited research that assessed the effects of resveratrol and pterostilbene on the intestinal microbiome in vivo [48,49] is available. In addition, nothing in the literature presently exists which directly measures the effects of these bioactive compounds on the mineral status, BBM morphology or functionality.

In the present study, we evaluated the effects of the intra-amniotic administration of resveratrol and pterostilbene on the relevant physiological, molecular and microbial parameters in vivo (Gallus gallus). Furthermore, the concentrations of the two stilbenes were determined in the red grape cultivars of three different species grown in the New York State Finger Lakes region: Vitis vinifera, Vitis labruscana and a French hybrid. This was done in order to associate the findings of this study with the practical dietary health benefits of grape and grape product consumption. Similarly, the synergistic group concentration was selected based on the realistic doses of resveratrol and pterostilbene found in nature [50].

Previous studies have shown that the intra-amniotic administration of prebiotics, soluble fiber and polyphenols affects the BBM morphology, specifically the villus surface area and goblet cell number, type and size [19,20,23,24,25,26]. In this study, the results indicate a significant increase in the duodenal villus length and diameter that was observed in all stilbenes treatment groups (5% resveratrol, 5% pterostilbene and synergistic). Furthermore, a significant increase in the goblet cell diameter was measured in the stilbene treatment groups relative to the controls (no injection, 18 MΩ H₂O and 5% inulin), which suggests an increased capacity for mucus production in the intestinal lumen. Goblet cells are mucus producing and secreting cells [51]. The mucin produced has the essential function of housing certain intestinal microbial populations [52] and contribute to nutrient digestion and absorption [53]. Changes in intestinal morphology are associated with the improvement in mineral status through enhanced bacterial composition and function in the gut. This observation demonstrates that stilbene administration induced enterocyte cell proliferation, which is considered a mechanical indicator of the BBM morphology, functionality and digestive and absorptive capabilities [28].

In the context of mineral absorption, as previously demonstrated [23,25,26,27,28,54], the bacterial fermentation activity led to increased production of short-chain fatty acids (SCFAs), which may indirectly result in increased mineral absorption. In the present study, there was downregulation in the relative expression of DcytB (Fe reductase) in all treatment groups compared with the controls (FIG. 1), this can suggest improved Fe bioavailability. To support this, we observed higher serum Fe and Zn concentrations and higher Zn storage in the liver samples of the synergistic group (Table 3), indicating that stilbene treatments have potential to improve the Fe and Zn metabolism-related pathways, and more studies are needed to confirm this observation. The low hemoglobin and high serum Fe concentrations observed in the synergistic group could be explained by the presence of more unbound serum Fe versus bound hemoglobin or Fe. However, a long-term feeding trial is warranted to further elucidate the possible mechanism of these effects. This is a novel finding that demonstrates improved mineral status and digestive capabilities in vivo. In accordance with this result, we observed upregulation in the gene expression of Δ-6-desaturase, a Zn-dependent enzyme, in the stilbene treatment groups. This observation suggests that the Zn's physiological status was improved and that Zn was present in a sufficient amount to support the appropriate function of this Zn-dependent enzyme [40,55].

In addition, upregulation in the gene expression of proteins involved with calcium and magnesium metabolism in the 5% pterostilbene and synergistic groups, relative to the 5% inulin control, was detected. This observation agrees with our hypothesis that the intra-amniotic administration of stilbenes beneficially modulates mineral absorption and therefore may improve BBM functionality.

The BBM functionality was appraised by measuring the gene expression of the functional proteins as biomarkers of BBM digestive and absorptive capabilities. The SGLT1 gene expression was upregulated in the stilbene treatment groups compared with the controls (FIG. 2). SGLT1 is a sodium-glucose transport protein responsible for dietary glucose absorption [56], and in agreement with our results, other studies have shown an increase in the gene expression of SGLT1 with the intra-amniotic administration of prebiotic-capacity soluble extracts [19,23]. In addition, it was previously suggested that SGLT1 activity contributes to the upregulation of the gene expression of intestinal mucin producing proteins via activation of a protein kinase C-dependent pathway that is controlled by the glucose uptake [57,58]. This mechanism can explain the increased goblet cell diameter in the stilbene treatment groups and further supports the improvement in BBM functionality.

Despite the low bioavailability of polyphenolic compounds in the gastrointestinal tract [59,60], stilbenes showed anti-inflammatory activity by the downregulation of IL-1β and TNF-α gene expressions compared with the inulin control (FIG. 2). Comparable results were demonstrated in vivo and in a study with humans [62], suggesting the potent action of these compounds on anti-inflammatory pathways. However, despite the antihypertensive activity demonstrated by stilbenes [42,63,64], we did not observe differences in the gene expressions of ACE and AT1R between the experimental groups. This may be due to acute exposure [65].

An additional objective of this study was to evaluate the specific effect of the stilbene treatments on the intestinal microbiome. A bidirectional relationship exists between the host and microbes that is vital to intestinal functionality and overall health [6,66,67]. We hypothesized that the stilbene treatments would alter the composition and function of the gut microbiome, similar to what previous studies found [49,68]. In the current study, the microbial analyses indicated that the 5% inulin treatment group differed in the distance metrics compared with the other treatment groups (FIG. 3B). The β diversity measured by Jaccard distances revealed that the treatment groups with stilbenes displayed strong clustering. Furthermore, the strong clustering between the 5% resveratrol and synergistic groups (FIG. 3C) indicated that their bacterial compositions were similar, which is unsurprising since these groups were compositionally similar. (The synergistic group received 47.5 mg mL⁻¹ of resveratrol and 2.5 mg mL⁻¹ of pterostilbene, and the resveratrol group received 50 mg mL⁻¹ of resveratrol) In addition, the microbial α diversity showed a lower bacterial abundance in the synergistic treatment group (FIG. 3A), which is an interesting result that merits future investigation.

Furthermore, we observed a lower abundance (<0.5%) of Actinobacteria in the synergistic group compared with the 5% inulin control. In addition, the trend observed in the synergistic group suggests a decreased abundance of Firmicutes (p=0.083). This could be considered a positive result, as the Firmicutes phylum is associated with being overweight and obesity in children and adults [69]. Previously, the consumption of resveratrol was shown to reduce the abundance of Firmicutes [70]. Certain members of this phylum produce trimethylamine-N-oxide (TMAO) as metabolites, which was recently linked to cardiovascular disease pathogenesis [71]. Since Firmicutes partially modulate the catabolism of choline and carnitine to TMAO [71,72], the suggested reduction in this phylum might be beneficial to the host.

Furthermore, a general taxonomic difference using LEfSe was observed between the 5% pterostilbene group and the no injection control (FIG. 5B), whereby the Bacteroidetes predominated in the 5% pterostilbene group. Bacteroidetes are SCFA-producing bacteria and mostly produce propionate and succinate [73]. We also observed that the abundance of the S24-7 family and Prevotella genus, belonging to the Bacteroidetes phylum, were enriched. Members of the S24-7 family, currently named Muribaculaceae [74], can ferment polysaccharides into SCFAs. We also observed that the Clostridiales order was enriched in the 5% pterostilbene group. This observation agrees with a previous clinical study by Calderón-Pérez et al. which recently demonstrated a positive correlation (p=0.037) between stilbenes and the abundance of the Clostridiales order. The elevation in SCFA-producing bacterial populations suggests an increased SCFA concentration in the lumen. As previously demonstrated, these SCFAs may lead to intestinal cellular (specifically enterocyte) proliferation [76]. This connection could explain the increase in the duodenal villi length, villi diameter and goblet cell diameter (Table 4 and Table 5) compared with the controls. Furthermore, by favoring bacterial fermentation, stilbenes affected the intestinal luminal pH, acidifying it and improving mineral (as Fe and Zn) solubility [17,19,21]. This could tie together with the improved Fe and Zn mineral status observed in this study (Table 3).

In addition, the gut microbiome data demonstrates how the detected and specific microbial profile within a treatment group is linked to the host's physiological status. The results show that the 5% pterostilbene group upregulated the pathways associated with cellular processes (e.g., DNA repair and recombination proteins, cell division, DNA replication and fatty acid biosynthesis) and metabolic processes (e.g., amino acid, sugar and nucleotide sugar metabolism, energy metabolism and glycolysis or gluconeogenesis metabolism) (FIGS. 6A-6D). We observed a significant difference in the KEGG microbial metabolic pathway analysis between the 5% pterostilbene group and all the other groups. This could be explained by the fact that pterostilbenes are highly absorbable (about 80%) or bioavailable when compared with resveratrol (about 20%) [11,12]. Additionally, the antioxidant and anti-inflammatory properties of stilbenes could beneficially modulate the gut microbiome [24], leading to the observed results.

### Summary

The Example at least demonstrates changes in the duodenal morphometric parameters after the intra-amniotic administration of stilbenes, which can improve micronutrient absorption. The 5% pterostilbene and synergistic treatments (4.75% resveratrol and 0.25% pterostilbene) improved BBM functionality, and as a result, led to an increase in the Fe and Zn serum concentrations. Although statistically significant modifications in the taxonomy of the cecal microbiota were not detected, upregulation in the SCFA-producing bacteria in the 5% pterostilbene group was observed. This modulation affected several bacterial metabolic pathways related to bacterial, cellular and metabolic processes. This data at least supports dietary stilbene consumption for improved gastrointestinal health and function, particularly in poultry.

### REFERENCES RELATED TO EXAMPLE 1

1. Yang, J.; Xiao, Y.-Y. Grape Phytochemicals and Associated. Health Benefits. Crit. Rev. Food Sci. Nutr. 2013, 53,1202-1225.
2. Georgiev, V.; Ananga, A.; Tsolova, V. Recent Advances and Uses of Grape Flavonoids as Nutraceuticals. Nutrients 2014, 6. 391-415.
3. Renaud, S.; de Lorgeril, M. Wine, alcohol, platelets, and the French paradox for coronary heart disease, Lancet 1992, 339,1523-1526.
4. Siemann, E. H.; Creasy, L. L. Concentration of the Phytoalexin Resveratrol in Wine. Am. J. Enol. Vitic, 1992, 43, 49-52.
5. Berman, A. Y.; Motechin, R. A.; Wiesenfeld, M. Y.; Holz, M. K. The therapeutic potential of resveratrol: A review of clinical trials. NPJ Precis. Oncol. 2017, 1, 35.
6. Cho, S.; Namkoong, K.; Shin, M.; Park, J.; Yang, E.; Ihm, J.; Thu. V. T.; Kim, H. K.; Han, J. Cardiovascular Protective Effects and Clinical Applications of Resveratrol. J. Med. Food 2017, 20, 323-334.
7. International Organisation of Vine and Wine. 2019 Statistical Report on World Vinviniculture;International Organisation of Vine and Wine: Paris, France, 2019.
8. Flamini, R.; De Rosso, M.; De Marchi, F.; Dalla Vedova, A.; Panighel, A.; Gardiman, M.; Maoz, I.; Bavaresco, L. An innovative approach to grape metabolomics: Stilbene profiling by suspect screening analysis. Metabolomics 2013, 9, 1243-1253,
9. Pezet, R.; Pont, V.; Cuenat, P. Method to determine resveratrol and pterostilbene in grape berries and wines using high-performance liquid chromatography and highly sensitive fluorimetric detection. J. Chromatogr. 01994, 663, 191-197.
10. Bavaresco, L.; Fregoni, C.; Cantu, E.; Trevisan, M. Stilbene compounds: From the grapevine to wine. Drugs Exp. Clin. Res. 1999, 25, 57-63.
11. Kapetanovic, I. M.; Muzzio, M.; Huang, Z.; Thompson, T. N.; McCormick, D. L. Pharmacokinetics, oral bioavailability, and metabolic profile of resveratrol and its dimethylether analog, pterostilbene, in rats. Cancer Chemother. Pharmacol. 2011, 68, 593-601.
12. Walle, T. Bioavailability of resveratrol. Ann. N. Y. Acad. Sci. 2011, 1215, 9-15.
13. Sreenivasulu, K,; Raghu, P.; Nair, K. M. Polyphenol-Rich Beverages Enhance Zinc Uptake and Metallothionein Expression in Caco-2 Cells. J. Food Sci. 2010, 75, H123-H128
14. Greger, J. L., Lyle, B. J. Iron, Copper and Zinc Metabolism of Rats Fed Various Levels and Types of Tea. J. Nutr. 1988, 118, 52-60.
15. Navarro, G., Martinez-Pinilla, E.; Ortiz, R.; Noé, V.; Ciudad, C. J.; Franco, R. Resveratrol and Related Stilbenoids, Nutraceutical/Dietary Complements with Health-Promoting Actions: Industrial Production, Safety, and the Search for Mode of Action. Compr. Rev. Food Sci. Food Saf. 2018, 17, 808-826.
16. Tako, E .; Glahn, R. P. Intra-amniotic administration and dietary inulin affect the iron status and intestinal functionality of iron-deficient broiler chickens. Poult. Sci. 2012, 91, 1361-1370.
17. Tako, E.; Glahn, R. P.; Knez, M.; Stangoulis, J. C. The effect of wheat prebiotics on the gut bacterial population and iron status of iron deficient broiler chickens. Nutr. J. 2014, 13, 58.
18. Tako, E.; Rutzke, M. A.; Glahn, R. P. Using the domestic chicken (Gallus gallus) as an in vivo model for iron bioavailability. Poult. Sci. 2010, 89, 514-521.
19. Pacifici, S.; Song, J.; Zhang, C.; Wang, Q.; Glahn, R.; Kolba, N.; Tako, E. Intra Amniotic Administration of Raffinose and Stachyose Affects the Intestinal Brush Border Functionality and Alters Gut Microflora Populations. Nutrients 2017, 9, 304
20. Hartono, K.; Reed, S.; Ankrah, N. A.; Glahn, R. P.; Tako, E. Alterations in gut microflora populations and brush border functionality following intra-amniotic daidzein administration. RSC Adv. 2015, 5, 6407-6412
21. Hou, T., Tako, E. The In Ovo Feeding Administration (Gallus gallus)—An Emerging In Vivo Approach to Assess Bioactive Compounds with Potential Nutritional Benefits. Nutrients 2018, 10, 418.
22. Reed, S.; Knez, M., Uzan, A.; Stangoulis, J. C. R.; Glahn, R. P.; Koren, O.; Tako, E. Alterations in the Gut (Gallus gallus) Microbiota Following the Consumption of Zinc Biofortified Wheat (Triticum aestivum)-Based Diet. J. Agric. Food Chem. 2018, 66, 6291-6299.
23. Wang, X.; Kolba, N., Liang, J., Tako, E. Alterations in gut microflora populations and brush border functionality following intra-amniotic administration (Gallus gallus) of wheat bran prebiotic extracts. Food Funct. 2019, 10, 4834-4843
24. Carboni, J.: Reed, S.; Kolba, N.; Eshel, A.; Koren, O.; Tako, E. Alterations in the Intestinal Morphology, Gut Microbiota, and Trace Mineral Status Following Intra-Amniotic Administration ((Gallus gallus) of Teff (Eragrostis tef) Seed Extracts. Nutrients 2020, 12, 3020.
25. Martino, H. S . D.; Kolba, N.; Tako, Yacon (Smallanthus sonchifolius) flour soluble extract improve intestinal bacterial populations, brush border membrane functionality and morphology in vivo (Gallus gallus). Food Res. Int, 2020, 137, 109705.
26. Pereira da Silva, B.; Kolba, N.; Martino, H. S. D.; Hart, J.; Tako, E. Soluble Extracts from Chia Seed (Salvia hispanica L.) Affect Brush Border Membrane Functionality, Morphology and Intestinal Bacterial Populations In Vivo (Gallus gallus). Nutrients 2019, 11, 2457.
27. Dias, D. M.; Kolba, N.; Hart, J. J.; Ma, M.; Sha, S. T.; Lakshmanan, N,; Nutti, M. R.; Martino, H. S. D., Glahn, R. P.; Tako, E. Soluble extracts from carioca beans (Phaseolus vulgaris L.) affect the gut microbiota and iron related brush border membrane protein expression in vivo (Gallus gallus). Food Res. Int. 2019, 123, 172-180.
28. Dias, D.; Kolba, N.; Binyamin, D.; Ziv, O.; Regini Nutti, M.; Martino, H.; Glahn, R.; Koren, O.; Tako, E. Iron Biofortified Carioca Bean (Phaseolus vulgaris L.) Based Brazilian Diet Delivers More Absorbable Iron and Affects the Gut Microbiota In Vivo (Gallus gallus). Nutrients 2018, 10, 1970.
29. Tako, E.; Blair, M. W.; Glahn, R. P. Biofortified red mottled beans (Phaseolus vulgaris L.) in a maize and bean diet provide more bioavailable iron than standard red mottled beans: Studies in poultry (Gallus gallus) and an in vitro digestion/Caco-2 model. Nutr. J. 2011, 10,113.
30. Tako, E.; Beebe, S. E.; Reed, S.; Hart, J. J.; Glahn, R. P. Polyphenolic compounds appear to limit the nutritional benefit of biofortified higher iron black bean (Phaseolus vulgaris L.). Nutr. J. 2014. 13, 28.
31. Caporaso, J. G.; Lauber, C. L., Walters, W. A.; Berg-Lyons, D.; Huntley, J.; Fierer, N.; Owens, S. M.; Betley, J.; Fraser, L.; Bauer, M.; et al. Ultra-high-throughput microbial community analysis on the Illumina HiSeg and MiSeq platforms. ISME J. 2012, 6, 1621-1624.
32. Bolyen, E.; Rideout, J. R.; Dillon, M. R.; Bokulich, N. A.; Abnet, C. C.; Al-Ghalith, G. A.; Alexander, H.; Alm, E. J.; Arumugam, M.; Asnicar, F.; et al. Reproducible, interactive, scalable and extensible microbiome data science using QIIME 2. Nat. Biotechnol. 2019, 37 , 852-857.
33. Callahan, B. J.; McMurdie, P. J.; Rosen, M. J.; Han, A. W.; Johnson, A. J. A., Holmes, S. P. DADA2: High-resolution sample inference from Illumina amplicon data. Nat. Methods 2016, 13, 581-583.

34. DeSantis, T. Z.; Hugenholtz, P.; Larsen, N.; Rojas, M.; Brodie, E. L.; Keller, K.; Huber, T.; Dalevi, D.; Hu, P.; Andersen, G. L. Greengenes, a Chimera-Checked 16S rRNA Gene Database and Workbench Compatible with ARB. Appl. Environ. Microbiol. 2006, 72, 5069-5072
35. Segata, N.; Izard, J.; Waldron, L.; Gevers, D.; Miropolsky, L., Garrett, W. S.; Huttenhower, C. Metagenomic biomarker discovery and explanation. Genome Biol. 2011, 12, R60.
36. Langille, M. G. I.; Zaneveld, J.; Caporaso, J. G.; McDonald, D.; Knights, D.; Reyes, J. A.; Clemente, J. C.; Burkepile, D. E.; Vega Thurber, R. L.; Knight, R., et al. Predictive functional profiling of microbial communities using 16S rRNA marker gene sequences. Nat. Biotechnol. 2013, 31, 814-821.
37. Tako, E.; Ferket, P.; Uni, Z. Changes in chicken intestinal zinc exporter mRNA expression and small intestinal functionality following intra-amniotic zinc-methionine administration. J. Nutr. Biochem. 2005, 16, 339-346.
38. Knez, M.; Stangoulis, J.; Glibetic, M.; Tako, E. The Linoleic Acid: Dihomo-γ-Linolenic Acid Ratio (LA:DGLA)—An Emerging Biomarker of Zn Status.Nutrients 2017, 9, 825.
39. Knez, M.; Tako, E.; Glahn, R. P., Kolba, N.; de Courcy-Ireland, E.; Stangoulis, J. C. R. Linoleic Acid: Dihomo-γ-Linolenic Acid Ratio Predicts the Efficacy of Zn-Biofortified Wheat in Chicken (Gallus gallus). J. Agric. Food Chem. 2018, 66, 1394-1400
40. Reed, S.; Qin, X.; Ran-Ressler, R.; Brenna, J.; Glahn, R.; Tako, E. Dietary Zinc Deficiency Affects Blood Linoleic Acid: Dihomo-γ-linolenic Acid (LA:DGLA) Ratio; a Sensitive Physiological Marker of Zinc Status in vivo (Gallus gallus). Nutrients 2014, 6, 1164-1180.
41. Mariat, D., Firmesse, O.; Levenez, F.; Guimarães, V.; Sokol, H.; Doré, J.; Corthier, G.; Furet, J.-P. The Firmicutes/Bacteroidetes ratio of the human microbiota changes with age. BMC Microbiol. 2009, 9, 123
42. Akinwumi, B.; Bordun, K.-A.; Anderson, H. Biological Activities of Stilbenoids. Int. J. Mol. Sci. 2018, 19, 792.
43. Salehi, B.; Mishra, A.; Nigam, M.; Sener, B.; Kilic, M.; Sharifi-Rad, M.; Fokou, P.; Martins, N.; Sharifi-Rad, J. Resveratrol: A Double-Edged Sword in Health Benefits. Biomedicines 2018, 6, 91.
44. Li, Y.-R.; Li, S.; Lin, C.-C. Effect of resveratrol and pterostilbene on aging and longevity. BioFactors 2018, 44, 69-82.
45. Jeandet, P.; Delaunois, B.; Conreux, A.; Donnez, D.; Nuzzo, V.; Cordelier, S.; Clément, C.; Courot, E. Biosynthesis, metabolism, molecular engineering, and biological functions of stilbene phytoalexins in plants. BioFactors 2010, 36, 331-341.
46. Rimando, A. M.; Cuendet, M.; Desmarchelier, C.; Mehta, R. G.; Pezzuto, J. M.; Duke, S. O. Cancer Chemopreventive and Antioxidant Activities of Pterostilbene, a Naturally Occurring Analogue of Resveratrol. J. Agric. Food Chem. 2002, 50, 3453-3457
47. Lv, M.; Liu, K., Fu, S.; Li, Z., Yu, X. Pterostilbene attenuates the inflammatory reaction induced by ischemia/reperfusion in rat heart. Mol. Med. Rep. 2015, 11, 724-728.
48. Etxeberria, U.; Hijona, E.; Aguirre, L.; Milagro, F. I.; Bujanda, L.; Rimando, A. M.; Martinez, J. A.; Portillo, M. P. Pterostilbene-induced changes in gut microbiota composition in relation to obesity. Mol. Nutr. Food Res. 2017, 61, 1500906.
49. Milton-Laskibar, I.; Marcos-Zambrano, L. J.; Gómez-Zorita, S.; Fernández-Quintela, A.; Carrillo de Santa Pau, E.; Martinez, J. A.; Portillo, M. P. Gut Microbiota Induced by Pterostilbene and Resveratrol in High-Fat-High-Fructose Fed Rats: Putative Role in Steatohepatitis Onset. Nutrients 2021, 13, 1738
50. Adrian, M.; Jeandet, P.; Breuil, A. C.; Levite, D.; Debord, S.; Bessis, R. Assay of resveratrol and derivative stilbenes in wines by direct injection high performance liquid chromatography. Am. J. Enol. Vitic. 2000, 51, 37-41
51. Birchenough, G. M. H.; Johansson, M. E.; Gustafsson, J. K.; Bergström, J. H.; Hansson, G. C. New developments in goblet cell mucus secretion and function. Mucosal Immunol. 2015, 8, 712-719.
52. Bergstrom, K.; Shan, X.; Casero, D.; Batushansky, A.; Lagishetty, V.; Jacobs, J. P.; Hoover, C., Kondo, Y.; Shao, B., Gao, L.; et al. Proximal colon-derived O-glycosylated mucus encapsulates and modulates the microbiota. Science 2020, 370, 467-472.
53. Montagne, L.; Piel, C.; Lalles, J. P. Effect of Diet on Mucin Kinetics and Composition: Nutrition and Health Implications. Nutr. Rev. 2004, 62, 105-114
54. Gomes, M. J. C.; Martino, H. S. D.; Tako, E. Effects of iron and zinc biofortified foods on gut microbiota in vivo (Gallus gallus): A systematic review. Nutrients 2021, 13, 189.
55. Liong, E .M.; McDonald, C. M.; Suh, J.; Westcott, J. L.; Wong, C. P.; Signorell, C.; King, J. C. Zinc-Biofortified Wheat Intake and Zinc Status Biomarkers in Men: Randomized Controlled Trial. J. Nutr. 2021, 151, 1817-1823.
56. Oguma, T.; Nakayama, K., Kuriyama, C.; Matsushita, Y.; Yoshida, K., Hikida, K., Obokata, N.; Tsuda-Tsukimoto, M.; Saito, A.; Arakawa, K.; et al. Intestinal Sodium Glucose Cotransporter 1 Inhibition Enhances Glucagon-Like Peptide-1 Secretion in Normal and Diabetic Rodents. J. Pharmacol. Exp. Ther. 2015, 354, 279-289.
57. Kellett, G. L. The facilitated component of intestinal glucose absorption. J. Physiol. 2001, 531, 585-595
58. Smirnov, A.; Tako, E.; Ferket, P. R.; Uni, Z. Mucin Gene Expression and Mucin Content in the Chicken Intestinal Goblet Cells Are Affected by In Ovo Feeding of Carbohydrates. Poult. Sci. 2006, 85, 669-673
59. Teng, H.; Chen, L. Polyphenols and bioavailability: An update. Crit. Rev. Food Sci. Nutr. 2019, 59, 2040-2051
60. Abdel-Moneim, A. E.; Shehata, A. M.; Alzahrani, S. O.; Shafi, M. E.; Mesalam, N. M.; Taha, A. E.; Swelum, A. A.; Arif, M.; Fayyaz, M.; Abd El-Hack, M. E. The role of polyphenols in poultry nutrition. J. Anim. Physiol. Anim. Nutr. 2020, 104, 1851-1866.
61. Liu, S.; Li, Y., Yi, F.; Liu, Q ; Chen, N.; He, X., He, C., Xiao, P. Resveratrol oligomers from Paeonia suffruticosa protect mice against cognitive dysfunction by regulating cholinergic, antioxidant and anti-inflammatory pathways. J. Ethnopharmacol. 2020, 260, 112983.
62. Ghanim, H.; Sia, C. L.; Korzeniewski, K.; Lohano, T.; Abuaysheh, S.; Marumganti, A.; Chaudhuri, A.; Dandona, P. A Resveratrol and Polyphenol Preparation Suppresses Oxidative and Inflammatory Stress Response to a High-Fat, High-Carbohydrate Meal. J. Clin. Endocrinol. Metab. 2011, 96, 1409-1414
63. Li, X.; Dai, Y.; Yan, S.; Shi, Y.; Li, J.; Liu, J.; Cha, L.; Mu, J. Resveratrol lowers blood pressure in spontaneously hypertensive rats via calcium-dependent endothelial NO production. Clin. Exp. Hypertens. 2016, 38, 287-293.
64. Riche, D. M.; Riche, K. D.; Blackshear, C. T.; McEwen, C. L.; Sherman, J. J.; Wofford, M. R.; Griswold, M. E. Pterostilbene on Metabolic Parameters: A Randomized, Double-Blind, and Placebo-Controlled Trial. Evid.-Based Complement. Altern. Med. 2014, 2014, 459165.
65. Uni, Z.; Noy, Y.; Sklan, D. Posthatch development of small intestinal function in the poult. Poult. Sci. 1999, 78, 215-222.
66. Cortés-Martin, A.; Selma, M. V.; Tomás-Barberán, F. A.; González-Sarrias, A.; Espín, J. C. Where to Look into the Puzzle of Polyphenols and Health? The Postbiotics and Gut Microbiota Associated with Human Metabotypes. Mol. Nutr. Food Res. 2020, 64, 1900952.
67. Shreiner, A. B.; Kao, J. Y.; Young, V. B. The gut microbiome in health and in disease. Curr. Opin. Gastroenterol.2015, 31, 69-75
68. Chen, Y.; Zhang, H.; Chen, Y.; Jia, P.; Ji, S.; Zhang, Y.; Wang, T. Resveratrol and its derivative pterostilbene ameliorate intestine injury in intrauterine growth-retarded weanling piglets by modulating redox status and gut microbiota. J. Anim. Sci. Biotechnol. 2021, 12, 70.
69. Da Silva, C. C.; Monteil, M. A.; Davis, E. M. Overweight and Obesity in Children Are Associated with an Abundance of Firmicutes and Reduction of Bifidobacterium in Their Gastrointestinal Microbiota. Child. Obes.2020, 16, 204-210.
70. Bird, J. K.; Raederstorff, D.; Weber, P.; Steinert, R. E. Cardiovascular and Antiobesity Effects of Resveratrol Mediated through the Gut Microbiota. Adv. Nutr. An. Int. Rev. J. 2017, 8, 839-849.
71. Romano, K. A.; Vivas, E. I.; Amador-Noguez, D.; Rey, F. E. Intestinal Microbiota Composition Modulates Choline Bioavailability from Diet and Accumulation of the Proatherogenic Metabolite Trimethylamine-N-Oxide. MBio 2015, 6.]
72. Wilson, A.; McLean, C.; Kim, R. B. Trimethylamine-N-oxide: A link between the gut microbiome, bile acid metabolism, and atherosclerosis. Curr. Opin. Lipidol. 2016, 27, 148-154.
73. Rios-Covian, D.; Salazar, N.; Gueimonde, M.; de los Reyes-Gavilan, C. G. Shaping the Metabolism of Intestinal Bacteroides Population through Diet to Improve Human Health. Front. Microbiol. 2017, 8, 376.
74. Lagkouvardos, I.; Lesker, T. R.; Hitch, T. C. A.; Gálvez, E. J. C.; Smit, N.; Neuhaus, K.; Wang, J.; Baines, J. F.; Abt, B.; Stecher, B.; et al. Sequence and cultivation study of Muribaculaceae reveals novel species, host preference, and functional potential of this yet undescribed family. Microbiome 2019, 7, 28
75. Calderón-Pérez, L., Llauradó, E.; Companys, J.; Pla-Pagà, L., Pedret, A.; Rubió, L.; Gosalbes, M. J.; Yuste, S.; Solà, R.; Valls, R. M. Interplay between dietary phenolic compound intake and the human gut microbiome in hypertension. A cross-sectional study. Food Chem. 2021, 344, 128567.
76. Sakata, T. Stimulatory effect of short-chain fatty acids on epithelial cell proliferation in the rat intestine: A possible explanation for trophic effects of fermentable fibre, gut microbes and luminal trophic factors. Br. J. Nutr. 1987, 58, 95-103.
77. Hou, T.; Kolba, N.; Glahn, R. P.; Tako, E. Intra-amniotic administration (Gallus gallus) of Cicer arietinum and Lens culinaris prebiotics extracts and duck egg white peptides affects calcium status and intestinal functionality. Nutrients 2017, 9, 785-803, doi: 10.3390/nu9070785.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method for improving avian gut health, the method comprising: administering, to an avian, optionally a poultry animal, an effective amount of a stilbene composition comprising one or more stilbenes.
2. The method of aspect 1, wherein the effective amount of the stilbene composition comprises 5-25 mg stilbene/kg of animal body weight.
3. The method of any one of aspects 1-2, wherein the stilbene composition comprises resveratrol, pterostilbene, piceatannol, or any combination thereof.
4. The method of any one of aspects 1-3, wherein the poultry animal is selected from the group consisting of chicken, turkey, duck, geese, quail, pheasant, emu, and ostrich.
5. The method of any one of aspects 1-3, wherein the avian is a wild bird species
6. The method of any one of aspects 1-6, wherein the effective amount of the stilbene composition is administered via oral, subcutaneous, intramuscular, or intra-amniotic administration.
7. The method of aspect 6, wherein the effective amount of the stilbene composition comprises resveratrol and wherein the resveratrol is administered at a concentration of 5-100 mg/ml.
8. The method of any one of aspects 6-7, wherein the effective amount of the stilbene composition comprises pterostilbene and wherein the pterostilbene is administered at a concentration of 5-100 mg/ml.
9. The method of any one of aspects 6-8, wherein effective amount of the stilbene composition comprises resveratrol and pterostilbene, wherein the resveratrol is administered at a concentration of 2.5-100 mg/ml, and wherein pterostilbene is administered at a concentration of 2.5-100 mg/ml.
10. The method of aspect 9, wherein resveratrol and pterostilbene are administered at a resveratrol to pterostilbene ratio of 95:5.
11. The method of any one of aspects 1-10, wherein the effective amount of the stilbene composition is administered via oral administration.
12. The method of aspect 11, wherein the oral administration comprises administering the stilbene in feed, in an aqueous solution, or both.
13. The method of aspect 12, wherein the aqueous solution is a water source for the avian.
14. The method of any one of aspects 1-13, wherein after administration of the effective amount of the stilbene composition the avian
   a. exhibits at least 5% greater villi surface area in the gut after administration;
   b. exhibits at least 5% greater goblet cell diameter;
   c. exhibits at least 5% increased liver iron concentrations;
   d. exhibits at least 5% increased liver zinc concentrations;
   e. exhibits at least 5% increased serum iron concentrations;
   f. exhibits at least 5% increased serum zinc concentrations;
   g. exhibits increased growth rate;
   h. exhibits increased bodyweight gain;
   i. exhibits decreased feed conversion ratio;
   j. comprises a healthy gut microbiome structure;
   k. or any combination thereof.
15. The method of any one of aspects 1-14, wherein the one or more stilbenes are isolated from grapes, optionally Vitis vinifera, peanuts, pistachios, blueberries, cranberries, tomato, cocoa, hop, rhubarb, strawberry, sugar cane, or any combination thereof.
16. An avian feed or aqueous formulation:
   comprising an effective amount of a stilbene composition comprising one or more stilbenes, wherein the amount of the stilbene composition is effective to improve avain gut health; and optionally a carrier.
17. The avian feed or aqueous formulation of aspect 16, wherein the stilbene composition comprises resveratrol, pterostilbene, piceatannol, or any combination thereof.
18. The avian feed or aqueous formulation of any one of aspects 16-17, the effective amount of the stilbene composition comprises 5-25 mg stilbene/kg of animal body weight.
19. The avian feed or aqueous formulation of any one of aspects 16-18, wherein the effective amount of the stilbene composition comprises resveratrol and wherein the resveratrol is administered at a concentration of 5-100 mg/ml.
20. The avian feed or aqueous formulation of any one of aspects 16-19, wherein effective amount of the stilbene composition comprises pterostilbene and wherein the pterostilbene is administered at a concentration of 5-100 mg/ml.
21. The avian feed or aqueous formulation of any one of aspects 16-20, wherein effective amount of the stilbene composition comprises resveratrol and pterostilbene, wherein the resveratrol is administered at a concentration of 2.5-100 mg/ml, and wherein pterostilbene is administered at a concentration of 2.5-100 mg/ml.
22. The avian feed or aqueous formulation of aspect 21, wherein resveratrol and pterostilbene are administered at a resveratrol to pterostilbene ratio of 95:5.
23. The avian feed or aqueous formulation of any one of aspects 16-22, wherein the avian feed is adapted for a poultry animal or a wild avian.
24. The avian feed or aqueous formulation of aspect 23, wherein the poultry animal is selected from the group consisting of chicken, turkey, duck, geese, quail, pheasant, emu, and ostrich.
25. The avian feed or aqueous formulation of any one of aspects 16-24, wherein the avian feed or aqueous formulation is adapted for dilution into a water source.
26. The avian feed or aqueous formulation of any one of aspects 16-25, wherein the effective amount of the stilbene composition is effective, in an avian, to
   a. increase villi surface area in the gut by at least 5%;
   b. increase greater goblet cell diameter by at least 5%;
   c. increase liver iron concentrations by at least 5%;
   d. increase liver zinc concentration by at least 5%;
   e. increase serum iron concentration by at least 5%;
   f. increase serum zinc concentration by at least 5%;
   g. increase growth rate of an avian;
   h. increase bodyweight gain;
   i. decrease feed conversion ratio;
   j. establish and/or maintain a healthy gut microbiome structure;
   k. or any combination thereof.
27. The avian feed or aqueous formulation of any one of aspects 16-26, wherein the one or more stilbenes are isolated from grapes, optionally Vitis vinifera, peanuts, pistachios, blueberries, cranberries, tomato, cocoa, hop, rhubarb, strawberry, sugar cane, or any combination thereof.

1-27. (canceled)

28. A method for improving gut health, the method comprising:
administering, to a subject in need thereof, a stilbene composition or formulation thereof, wherein the stilbene composition comprises an effective amount of one or more stilbenes, wherein the effective amount of the one or more stilbenes is 5-25 mg stilbene/kg of body weight of the subject in need thereof.

29. The method of claim 28, wherein the stilbene composition comprises resveratrol, pterostilbene, piceatannol, or any combination thereof.

30. The method of claim 28, wherein the subject in need thereof is avian.

31. The method of claim 30, wherein the avian is poultry or a wild bird species.

32. The method of claim 31, wherein the poultry is selected from the group consisting of chicken, turkey, duck, geese, quail, pheasant, emu, and ostrich.

33. The method of claim 28, wherein the subject in need thereof is a human or non-human animal.

34. The method of claim 28, wherein (a) the stilbene composition comprises resveratrol at a concentration of 5-100 mg/ml; (b) the stilbene composition comprises pterostilbene at a concentration of 5-100 mg/ml; or (c) the stilbene composition comprises resveratrol at a concentration of 2.5-100 mg/ml and pterostilbene at a concentration of 2.5-100 mg/ml.

35. The method of claim 28, wherein the stilbene composition comprises resveratrol and pterostilbene at a resveratrol to pterostilbene ratio of 95:5.

36. The method of claim 28, wherein the effective amount of the stilbene composition is administered via oral, subcutaneous, intramuscular, in ovo, or intra-amniotic administration.

37. The method of claim 36, wherein the oral administration comprises administering the stilbene composition in (a) a feed or in a foodstuff; (b) in a liquid formulation; or both (a) and (b).

38. The method of claim 37, wherein the liquid solution is a water source for the subject in need thereof.

39. The method of claim 28, wherein after administration of the stilbene composition the subject in need thereof.
a. exhibits at least 5% greater villi surface area in the gut after administration;
b. exhibits at least 5% greater goblet cell diameter;
c. exhibits at least 5% increased liver iron concentrations;
d. exhibits at least 5% increased liver zinc concentrations;
e. exhibits at least 5% increased serum iron concentrations;
f. exhibits at least 5% increased serum zinc concentrations;
g. exhibits increased growth rate;
h. exhibits increased bodyweight gain;
i. exhibits decreased feed conversion ratio;
j. comprises a healthy gut microbiome structure;
k. or any combination thereof.

40. The method of claim 28, wherein the one or more stilbenes are isolated from grapes, optionally Vitis vinifera, peanuts, pistachios, blueberries, cranberries, tomato, cocoa, hop, rhubarb, strawberry, sugar cane, or any combination thereof; or wherein the composition comprises an amount of grapes, optionally Vitis vinifera, peanuts, pistachios, blueberries, cranberries, tomato, cocoa, hop, rhubarb, strawberry, sugar cane, or any combination thereof.

41. A formulation comprising:
comprising a stilbene composition comprising an effective amount of one or more stilbenes, wherein the effective amount of the one or more stilbenes is 5-25 mg stilbene/kg of body weight of a subject to which formulation is administered or is consumed by; and
a carrier.

42. The formulation of claim 41, wherein the stilbene composition comprises resveratrol, pterostilbene, piceatannol, or any combination thereof.

43. The formulation of claim 41, wherein (a) the stilbene composition comprises resveratrol at a concentration of 5-100 mg/ml; (b) the stilbene composition comprises pterostilbene at a concentration of 5-100 mg/ml; or (c) the stilbene composition comprises resveratrol at a concentration of 2.5-100 mg/ml and pterostilbene at a concentration of 2.5-100 mg/ml.

44. The formulation of claim 43, wherein the stilbene composition comprises resveratrol and pterostilbene at a resveratrol to pterostilbene ratio of 95:5.

45. The formulation of claim 43, wherein the formulation is a feed, a foodstuff, or liquid formulation.

46. The formulation of claim 43, wherein the effective amount of the stilbene composition is effective, in the subject, to
a. increase villi surface area in the gut by at least 5%;
b. increase greater goblet cell diameter by at least 5%;
c. increase liver iron concentrations by at least 5%;
d. increase liver zinc concentration by at least 5%;
e. increase serum iron concentration by at least 5%;
f. increase serum zinc concentration by at least 5%;
g. increase growth rate of an avian;
h. increase bodyweight gain;
i. decrease feed conversion ratio;
j. establish and/or maintain a healthy gut microbiome structure;
k. or any combination thereof.

47. The formulation of claim 43, wherein the one or more stilbenes are isolated from grapes, optionally Vitis vinifera, peanuts, pistachios, blueberries, cranberries, tomato, cocoa, hop, rhubarb, strawberry, sugar cane, or any combination thereof; or wherein the composition comprises an amount of grapes, optionally Vitis vinifera, peanuts, pistachios, blueberries, cranberries, tomato, cocoa, hop, rhubarb, strawberry, sugar cane, or any combination thereof.
